(19) United States
(12) Patent Application Publication
(10) Pub. No.: US 2008/0286202 A1
(43) Pub. Date: Nov. 20, 2008

(54) Method of Diagnosing Prodromal Forms of Diseases Associated With Amyloid Deposition

(75) Inventors: William E. Klunk, Pittsburgh, PA (US); Chester A. Mathis Jr., Pittsburgh, PA (US)

Correspondence Address: FOLEY AND LARDNER LLP;SUITE 500, 3000 K STREET NW, WASHINGTON, DC, 20007, US

(73) Assignee: UNIVERSITY OF PITTSBURGH, Pittsburgh, PA (US)

(21) Appl. No.: 11/666,084

(22) PCT Filed: Jul. 1, 2005
(86) PCT No.: PCT/US05/23618

§ 371 Date: Oct. 4, 2007

Related U.S. Application Data
(60) Provisional application No. 60/584,495, filed on Jul. 2, 2004.

(51) Int. Cl. A61K 51/04; A61K 49/00; A61K 49/04; C07D 277/66; A61K 49/10

(52) U.S. Cl. 424/1.81; 424/9.1; 424/9.3; 424/9.44; 548/178

(57) ABSTRACT

A method of identifying a patient as prodromal to a disease associated with amyloid deposition by imaging techniques is provided. In addition, a method of identifying amyloid deposition diseases in patients who present with a dementing disorder of questionable etiology by imaging techniques is provided. The methods discloses substances which are used for imaging and generating data which can be used to determine progress of an asymptomatic patient to a disease associated with amyloid deposition, or to identify amyloid deposition diseases in patients who present with a dementing disorder of questionable etiology.

### FIELD OF THE INVENTION

The present invention relates generally to the field of diagnosis in a patient exhibiting signs of clinical dementia. In particular, the application is directed to a method for imaging areas of amyloid deposition in patients exhibiting clinical signs of dementia in pre-diagnosed states, such as mild cognitive impairment (MC1), or in a dementing disorder of questionable etiology and comparing the data obtained with normative levels in a control subject.

### BACKGROUND OF THE INVENTION

A. Diseases Associated with Amyloid Deposition

A condition closely related to Alzheimer's Disease (AD) is characterized by either isolated memory impairment or impairment in several cognitive domains, but not of sufficient severity to meet diagnostic criteria for Alzheimer's disease. This condition has been termed mild cognitive impairment and may represent a prodromal phase of AD. Mild cognitive impairment is defined as an intermediate or transitional state from a normal cognitive state to dementia. Subjects with a mild cognitive impairment (MC1) typically have a memory impairment beyond that expected for age and education yet are not demented.

There is some indication that patients diagnosed as mild cognitive impairment will progress to AD. There is also indications that mild cognitive impairment may represent a complex heterogeneous condition and that some patients with mild cognitive impairment will not develop AD or other dementing disorders.

There have been volumes of interest in discerning the boundary of dementia to AD. Most of the interest deals with a boundary or transitional state between normal aging and dementia, or more specifically, Alzheimer disease (AD). Reviews of several studies have indicated that these individuals are at an increased risk for developing AD ranging from 1% to 25% per year. The variability in these rates likely reflects differing diagnostic criteria, measurement instruments, and small sample sizes. See Dawe et al., Int'l J. Geriatr. Psychiatry 7: 473 (1992).

Patients diagnosed with an MCI are also becoming of interest for treatment trials. The Alzheimer's Disease Cooperative Study, which is a National Institute on Aging consortium of Alzheimer's Disease research groups, is embarking on a multicenter trial of agents intended to alter the progression of patients with MCI to AD. See Grundman et al., Neurology, 1996, A403.

Questions can be raised as to the diagnostic criteria for MC1. Some investigators believe that virtually all these patients with mild disease have AD neuropathologically, and, therefore, this may not be a useful distinction. See Morris et al., Neurology 41: 469 (1991). Others note that, while many of these patients progress to AD, not all do and, consequently, that the distinction is an important one. See Grundman, ibid; Petersen et al., JAMA 273: 1274 (1995); Petersen et al., Ann NY Acad. Sci. 802: 58 (1996).

AD is believed to afflict some 4 million Americans and perhaps 20-30 million people worldwide. AD is recognized as a major public health problem in developed nations.

AD is a neurodegenerative illness characterized by memory loss and other cognitive deficits. McKhann et al., Neurology 34: 939 (1984). It is the most common cause of dementia in the United States. AD can strike persons as young as 40-50 years of age, yet, because the presence of the disease is difficult to determine without dangerous brain biopsy, the time of onset is unknown. The prevalence of AD increases with age, with estimates of the affected population reaching as high as 40-50% by ages 85-90. Evans et al., JAMA 262: 2551 (1989); Katzman, Neurology 43: 13 (1993).

Neuropathologically, AD is characterized by the presence of neuritic plaques (NP), neurofibrillary tangles (NFT), and neuronal loss, along with a variety of other findings. Mann, Mech. Ageing Dev. 31: 213 (1985). Post-mortem slices of brain tissue of victims of AD exhibit the presence of amyloid in the form of proteinaceous extracellular cores of the neuritic plaques that are characteristic of AD. The amyloid cores of these neuritic plaques are composed of a protein called the β-amyloid (Aβ) that is arranged in a predominately beta-pleated sheet configuration.

B. Imaging of Amyloid Deposits

The first study to report human, in vivo amyloid imaging with a thioflavin derivative was presented in preliminary form by Engler et al., Neurobiol. Aging 23(1S): S429 (2002). Klunk et al., Annals of Neurology, 55: 306 (2004), provided a more detailed account. This study used a carbon-11-labeled benzothiazole derivative of the amyloid dye thioflavin-T, termed PIB (for Pittsburgh Compound-B).

As noted, the neuropathology of Alzheimer's disease frequently includes amyloid plaques, neurofibrillary tangles (Mirra et al., Neurology 41:479 (1991)), and α-synuclein deposits in the form of Lewy bodies or threads making AD a “triple amyloidosis” (Trojanowski et al., Neuromuscular Disorders 4:1 (2003)). Studies have been performed that address the relative specificity of PIB for AP amyloid deposits in light of the potential for co-deposition of NFT and α-synuclein.

At the nanomolar concentrations attainable in human Positron Emission Tomography (PET) studies, PIB and related benzothiazole derivatives bind to homogenates of plaque- and cerebrovascular amyloid-containing AD brain frontal cortex at 10-fold higher levels than the background binding observed in amyloid-free control brain frontal cortex. Klunk et al., J. Neurosci. 23: 2086 (2003).

That certain benzothiazole compounds can cross the blood brain barrier and target amyloid plaques points up a possibility of using the imaging agents to diagnose diseases associated with amyloid deposition prior to clinical symptoms. The ability to diagnose AD early and even to predict it, based on criteria seen in patients clinically diagnosed with mild cognitive impairment or another dementing disorder of questionable etiology, would enhance the care and maintenance of the elderly population afflicted with AD. To date, however, no definitive criteria have been established that would permit a physician accurately to determine onset of an amyloid deposition disease in an asymptomatic patient.

### SUMMARY OF THE INVENTION

One embodiment of the present invention relates to a method of identifying a patient as prodromal to a disease associated with amyloid deposition comprising:

(A) administering to the patient, who is presenting with signs of clinical dementia or clinical signs of a mild cognitive impairment, a compound of the following formula:

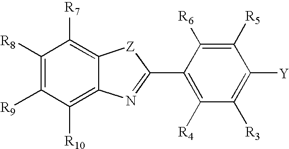

wherein
(i) Z is S, NR′, O or C(R′)₂, such that when Z is C(R′)₂, the tautomeric form of the heterocyclic ring may form an indole:

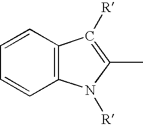

wherein R′ is H or a lower alkyl group,
   (ii) Y is NR¹R², OR² or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R¹ is H or a lower alkyl group),
(iv) R² is selected from the group consisting of H, a lower alkyl group, (CH₂)ₙOR′ (wherein n=1, 2 or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R′ is H or a lower alkyl group),
(v) each R³-R¹⁰ independently is selected from the group consisting of H, F, Cl, Br, I, a lower alkyl group, (CH₂)ₙOR′ (wherein n=1, 2 or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, with the phenyl substituents chosen from any of the non-phenyl substituents defined for R¹-R¹⁰ and wherein R′ is H or a lower alkyl group), a tri-alkyl tin and a chelating group (with or without a chelated metal group) of the form W-L or V—W-L, wherein V is selected from the group consisting of —COO—, —CO—, —CH₂O— and —CH₂NH—; W is —(CH₂)ₙ (where n=0, 1, 2, 3, 4 or 5) and L is:

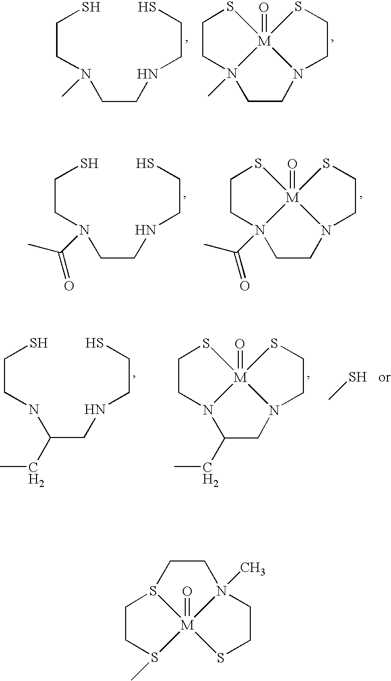

wherein M is selected from the group consisting of Tc and Re; and radiolabelled derivatives and pharmaceutically acceptable salts thereof, where at least one of the substituent moieties comprises a detectable label;

then

(B) imaging said patient to obtain data; and

(C) analyzing said data to ascertain amyloid levels in said patient with reference to a normative level, thereby identifying said patient as prodromal to a disease associated with amyloid deposition. In one aspect of the invention, the patient is diagnosed with mild cognitive impairment. In another aspect of the invention, the amyloid disease is Alzheimer's disease.

The detectable label includes any atom or moiety which can be detected using an imaging technique known to those skilled in the art. Typically, the detectable label is selected from the group consisting of ³H, ¹³¹I, ¹²⁵I, ¹²³I, ⁷⁶Br, ⁷⁵Br, ¹⁸F, CH₂—CH₂—X*, O—CH₂—CH₂—X*, CH₂—CH₂—CH₂—X*, O—CH₂—CH₂—CH₂—X* (wherein X*=¹³¹I, ¹²³I, ⁷⁶Br, ⁷⁵Br or ¹⁸F), ¹⁹F, ¹²⁵I, a carbon-containing substituent selected from the group consisting of lower alkyl, (CH₂)nOR′, CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, (C═O)N(R′)₂, O(CO)R′, COOR′, CR′═CR′—Rₚₕ and CR₂′—CR₂′—Rₚₕ wherein at least one carbon is ¹¹C, ¹³C or ¹⁴C and a chelating group (with chelated metal group) of the form W-L* or V—W-L*, wherein V is selected from the group consisting of —COO—, —CO—, —CH₂O— and —CH₂NH—; W is —(CH₂)ₙ where n=0, 1, 2, 3, 4, or 5; and L* is:

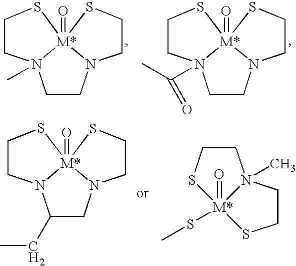

wherein M* is ^{99m}Tc. In a preferred embodiment, the detectable label is a radiolabel.

Using the same protocol, one can compare data obtained from the imaging techniques applied to the patients in order to:

define a dementing disorder of questionable etiology as being caused by an amyloid deposition disease;

distinguish Alzheimer's disease from frontotemporal dementia;

monitor a patient to determine onset of Alzheimer's disease;

diagnose Alzheimer's disease in a patient clinically diagnosed with mild cognitive impairment;

identify a patient as prodromal to Alzheimer's disease;

identify a patient as having a disease associated with an amyloid deposition disorder wherein the patient is presenting with a dementing disorder of questionable etiology or

identify a patient as having Alzheimer's disease wherein the patient is presenting with a dementing disorder of questionable etiology.

In one embodiment, the imaging of the inventive methodology is selected from the group consisting of gamma imaging, magnetic resonance imaging and magnetic resonance spectroscopy. In one aspect of this embodiment, the imaging is done by gamma imaging, and the gamma imaging is PET or SPECT.

In a preferred embodiment, the compound of Formula (I) is:

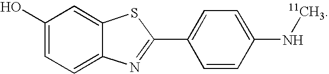

In particular, the above compounds contains a C-11 label.

The invention also provide methodology for identifying a patient as prodromal to a disease associated with amyloid deposition or presenting with a dementing disorder of questionable etiology previously undiagnosed with AD.

In a preferred embodiment, the amyloid deposition disorder is an amyloid plaque deposition disorder.

### BRIEF DESCRIPTION OF THE DRAWINGS

FIG. 1 shows Positron Emission Tomography (PET) brain scans using average Standardized Uptake Values (SUV) in a control patient, two patients clinically diagnosed as mild cognitive impairment patients and an Alzheimer's Disease (AD) patient.

FIG. 2 shows a graph of the correlation with rCMRglc in parietal cortex PIB SUV values.

FIG. 3 shows Logan DVR values determined in control, AD and MCI subjects.

FIG. 4 shows examples of PIB input functions and fraction of unmetabolized PIB in plasma.

FIG. 5 shows PIB time-activity data measured in controls and patients.

FIG. 6 shows a comparison of modeling methods of the calculation of Logan DVR values in control and AD subjects.

FIG. 7 shows Logan DVR images from control, MCI and AD. The MCI-1 subject had shown progressive deterioration, while MCI-2 has had a very stable, mild memory loss. The images (Logan ART 90 min) show the similarity of MCI-2 to controls and MCI-1 to the AD subjects.

FIG. 8 shows PIB SUV images generated AD-2 (left) and C-1 subjects (right) in coronal (top), transaxial (center) and sagittal views (bottom).

FIG. 9 shows image maps of the Logan PIB DVR (ART 90) (top), MR images (middle) and glucose metabolism (bottom) measured in a control, MCI (MCI-1), and AD subject. Greater PIB retention is evident in the cortex of the AD and MCI subjects, relative to the control. The map of glucose metabolism shows lower parietal metabolism for the AD subject.

FIG. 10 shows test/Re-Test Studies in Five Subjects. The test/re-test variability is expressed as the mean ±SD of the absolute value of the difference between the first and second PIB study within 21 days.

FIG. 11 shows stability of Logan DVR values determined with cerebellar input with reference to cerebellum. Five subjects returned for a re-test study within 21 days of their initial scan. Shown are five posterior cingulate cortex test/re-test DVR pairs determined using 60 min of data and cerebellar input.

FIG. 12 shows population average unchanged fraction of PIB in plasma that was determined in 16 subjects with complete metabolite data (A). Average (n=24) PIB input functions normalized for injected dose and body mass (% ID*kg/g) derived from both external arterial sampling and a carotid volume of interest (B). The population average PIB unchanged fraction was used to correct the carotid time-activity curve for metabolism, while individual data was applied to the total plasma radioactivity measurements to perform metabolite correction of the arterial input function.

FIG. 13 shows average (±1 SD) brain radioactivity concentrations normalized for injected dose and body mass (% ID*kg/g) following the injection of PIB. Shown are posterior cingulate gyrus (A) and cerebellar (B) regions for AD (n=6) and control (n=8) subjects, as well as the ratio of posterior cingulate gyrus and cerebellar radioactivity concentration (C).

FIG. 14 shows the outcome measures for individual AD (n=6, red), MCI (n=10, green) and control (n=8, blue) subjects across all methods of analysis for posterior cingulate gyrus (A) and frontal cortex (B). The outcome measure represented for all methods is the DVR with the exception of the SUVR90 and SUVR60 methods, for which the tissue:cerebellar ratio over 40-60 min or 40-90 min is shown. The numbered circles represent the individual subjects (see table 2), while the colored bars denote the range of values within the group. Subjects with overlapping values are placed adjacent to one another.

FIG. 15 shows parametric images of the Logan DVR using 90 min of emission data and either arterial data (ART90; top) or cerebellar tissue (CER90; bottom) as input. Shown are a young control (C-4), a control with detectable amyloid deposition in frontal cortex (C-2), an amyloid-negative MCI subject (M-2), an MC1 subject with intermediate levels of PIB retention (M-10), an amyloid-positive MCI subject with levels of PIB retention characteristic of AD (M-4), and a typical AD subject (A-2).

FIG. 16 shows bias and correlation measures of the various simplified methods with ART90. (A) Box plot showing the % bias in the simplified outcome measures relative to ART90 in the PCG. Subjects were divided into high-binding (ART90 PCG DVR>1.8) and low-binding (ART90 PCG DVR<1.8) groups to determine whether or not methodological bias was consistent across the spectrum of PIB retention for all simplified analysis methods. The boxes denote the interquartile range (50% of subjects) and median value (solid line), while the box whiskers indicate the 10^{th} and 90^{th} percentile. Individual subject values are represented by open circles. (B) Slopes of linear correlations between ART90 and the simplified methods. (C) Coefficient of determination (r²) for the correlations between ART90 and the simplified methods.

FIG. 17 depicts (A) a graph showing the correlation of ART90 and CAR90 (open circles, solid line) and SUVR90 and CER90 (filled circles, solid line) outcome measures; and (B) a graph showing the correlation between ART90 and CER60 Milled squares, solid line) and ART90 and CER60 (open squares, solid line) outcome measures. The equation describing the linear regression is shown for each comparison in the form y=mx+b as well as the coefficient of determination (r²). The thin dashed line in both graphs represents the line of unity.

### DETAILED DESCRIPTION OF THE PREFERRED EMBODIMENTS

The present inventors have determined that certain thioflavin compounds can be used to image amyloid deposits in the brains of patients who do not meet criteria for the diagnosis of AD, such as patients presenting with clinical signs of dementia or patients with a mild cognitive impairment, including patients presenting a dementing disorder of questionable etiology, where data from amyloid imaging of patients reveals that certain amyloid deposits are a premonitory symptom of AD or another amyloid deposition disorder.

The present invention is directed to a method of identifying a patient as prodromal to a standard clinical diagnosis of a amyloid deposition disease. The method involves the use of amyloid imaging agents to obtain quantitative and qualitative data from a patient. Quantitative and qualitative amyloid imaging, in accordance with the present invention, should allow for earlier and more accurate diagnosis of amyloid deposit diseases, and should aid in the development of anti-amyloid therapies. The target patient for this methodology is a patient presenting signs of clinical dementia or a patient exhibiting clinical signs of mild cognitive impairment.

One skilled in the art would recognize that the practitioner may apply different criteria for a determination of signs of clinical dementia. Such criteria include, but are not limited to Diagnostic and Statistical Manual of Mental Disorders, third edition (DSM-III) Alzheimer's Disease Diagnostic and Treatment Center (ADDTC), International Statistical Classification of Diseases, 10^{th} Revision (ICD-10), National Institute of Neurological Disorders and Stroke-Association Internationale pour 1a Recherche et l'Enseignment en Neurosciences (NINDS-AIREN) and Diagnostic and Statistical Manual of Mental Disorders, Fourth Edition (DSM-IV). See Pohjasvaara et al., Stroke, 2000 31; 2952-2957.

Clinical characterization of a patient as mild cognitive impairment is well within the skill of the practitioner. Such testing of a patient to elucidate such a condition involves performing a series of mental tests. The methods for clinical diagnosis are widely reviewed and are discussed in, e.g., Petersen et al., Arch. Neurol. Vol. 56, p 303-308, March 1999.

Based on clinical testing alone, subjects identified with MCI may convert to a diagnosis of AD (at a rate of about 10-15% per year), remain MC1, or revert to a diagnosis of “normal” (10-15% per year).

Larrieu, S, Letenneur, L, Orgogozo, J M, Fabrigoule, C, Amieva, H, Le, C, Barberger-Gateau, P, Dartigues, J F (1926) Incidence and outcome of mild cognitive impairment in a population-based prospective cohort. Neurology. 59:1594-1599.

Therefore, there is considerable prognostic uncertainty associated with this clinical diagnosis. The ability to identify the presence or absence of brain amyloid deposition in a subject clinically diagnosed with MCI has the potential to greatly increase the accuracy of prognosis for conversion to AD.

The category of diseases associated with amyloid deposition includes but is not limited to Alzheimer's Disease, Down's Syndrome, Type 2 diabetes mellitus, hereditary cerebral hemorrhage amyloidosis (Dutch), amyloid A (reactive), secondary amyloidosis, familial Mediterranean fever, familial amyloid nephropathy with urticaria and deafness (Muckle-wells Syndrome), amyloid lambda L-chain or amyloid kappa L-chain (idiopathic, myeloma or macroglobulinemia-associated) A beta 2M (chronic hemodialysis), ATTR (familial amyloid polyneuropathy (Portuguese, Japanese, Swedish)), familial amyloid cardiomyopathy (Danish), isolated cardiac amyloid, systemic senile amyloidoses, AIAPP or amylin insulinoma, atrial naturetic factor (isolated atrial amyloid), procalcitonin (medullary carcinoma of the thyroid), gelsolin (familial amyloidosis (Finnish)), cystatin C (hereditary cerebral hemorrhage with amyloidosis (Icelandic)), AApo-A-I (familial amyloidotic polyneuropathy-Iowa), AApo-A-II (accelerated senescence in mice), fibrinogen-associated amyloid; and Asor or Pr P-27 (scrapie, Creutzfeld Jacob disease, Gertsmann-Straussler-Scheinker syndrome, bovine spongiform encephalitis) or in cases of persons who are homozygous for the apolipoprotein E4 allele, and the condition associated with homozygosity for the apolipoprotein E4 allele or Huntington's disease. Preferably the disease associated with amyloid deposition is a amyloid plaque deposition disease. Preferably, the disease associated with amyloid deposition is AD.

According to the invention, a basic methodology of identifying a patient as prodromal to an amyloid deposition disease entails:

(A) administering to the patient, who is presenting with signs of clinical dementia or presenting with clinical signs of a mild cognitive impairment, in need thereof an effective amount of compound of the following formula:

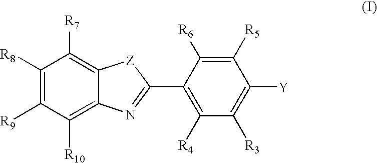

wherein

(i) Z is S, NR′, O or C(R′)₂, such that when Z is C(R′)₂, the tautomeric form of the heterocyclic ring may form an indole:

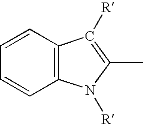

wherein R′ is H or a lower alkyl group,

(ii) Y is NR¹R², OR², or SR²,

(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from any of the non-phenyl substituents defined below for R³-R¹⁰ and R¹ is H or a lower alkyl group);

(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from any of the non-phenyl substituents defined below for R³-R¹⁰ and R′ is H or a lower alkyl group);

(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁰ and wherein R′ is H or a lower alkyl group) and a tri-alkyl tin;

alternatively, one of R³-R¹⁰ may be a chelating group (with or without a chelated metal group) of the form W-L or V—W-L, wherein V is selected from the group consisting of —COO—, —CO—, —CH₂O— and —CH₂NH—; W is —(CH₂)ₙ where n=0, 1, 2, 3, 4, or 5; and L is:

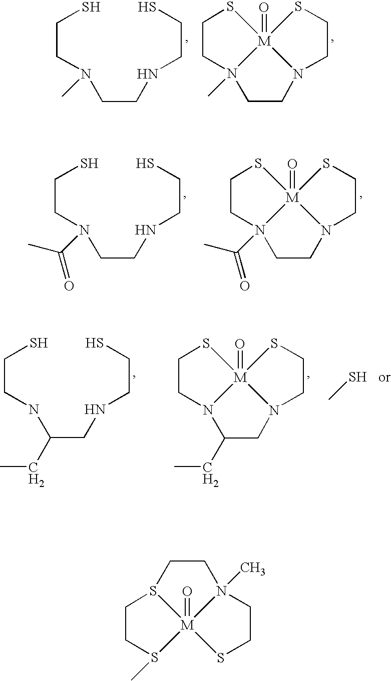

wherein M is selected from the group consisting of Tc and Re;

and radiolabeled derivatives and pharmaceutically acceptable salts thereof, where at least one of the substituent moieties comprises a detectable label;

(B) imaging said patient to obtain data and

(C) analyzing said data to ascertain amyloid levels in said patient with reference to a normative patient.

One embodiment relates to a method for diagnosing a patient presenting with a dementing disorder of questionable etiology. This method would involve determining if dementias of questionable etiology are likely to be AD or another amyloid deposition disorder based on the finding of amyloid deposition. This method would involve administering to a patient a compound of Formula (I) or (II) or one of structures 1-45, imaging the patient to obtain data and determining if the dementia of questionable etiology is AD based on the finding of amyloid deposition.

Another embodiment is a method of manufacturing a medicament for identifying a patient as prodromal to an amyloid deposition disease as described in any of the foregoing or following embodiments. The method comprises combining a compound according to formula I or II or one of structures 1-45 described herein, with a pharmaceutical carrier to form the medicament.

Yet another embodiment is a method of manufacturing a medicament for diagnosing a patient presenting with a dementing disorder of questionable etiology as set forth in any of the foregoing or following embodiments. The method comprises combining a compound according to formula I or II or one of structures 1-45 described herein, with a pharmaceutical carrier to form the medicament.

The term “dementing disorder of questionable etiology” refers to the condition in which a person presents for clinical evaluation (which may consist of neurological, psychiatric, medical and neuropsychological evaluations commonly employed by those skilled in the art of diagnosing persons with dementing disorders) and, after that clinical evaluation, the evaluator finds evidence that some dementing disorder may be present (based on evidence of subjective memory complaints, description of memory complaints by informants familiar with the persons deviation from normal functioning, or poor performance on neuropsychological and clinical tests commonly used by those skilled in the art), but, can not find sufficient evidence for any single clinically defined dementing disorder (such as AD, frontotemporal dementia, Dementia with Lewy Bodies, Vascular dementia, pseudodementia due to Major Depression, Creutzfeld Jacob disease and others known to those skilled in the art) or finds that the person shows evidence of more than one single dementing disorder to the degree that the distinction between these two (or more) dementing disorders is questionable in this person.

This aspect of the invention employs amyloid imaging agents which, in conjunction with non-invasive neuroimaging techniques such as magnetic resonance spectroscopy (MRS) or imaging (MRI), or gamma imaging such as positron emission tomography (PET) or single-photon emission computed tomography (SPECT), are used to quantify amyloid deposition in vivo. These imaging techniques acquire data on many brain regions. Quantitation on specific regions is achieved by delineating “regions of interest or ROI”.

Pursuant to the invention, data obtained from patients using one of the imaging techniques mentioned above can be compared to data from normative patients with a conclusion based on criteria which distinguish the patient as prodromal to a standard clinical diagnosis of an amyloid deposition disease.

Using the same protocol, one can compare data obtained from the imaging techniques applied to the patients in order to:

define a dementing disorder of questionable etiology as being caused by an amyloid deposition disease;

distinguish Alzheimer's disease from frontotemporal dementia;

monitor a patient to determine onset of Alzheimer's disease;

diagnose Alzheimer's disease in a patient clinically diagnosed with mild cognitive impairment;

identify a patient as prodromal to Alzheimer's disease;

identify a patient as having a disease associated with an amyloid deposition disorder wherein the patient is presenting with a dementing disorder of questionable etiology or identify a patient as having Alzheimer's disease wherein the patient is presenting with a dementing disorder of questionable etiology.

### Amyloid Imaging Agents

An amyloid imaging agent suitable for the present invention is any compound of formula (I), described above.

In some embodiments, the amyloid imaging agent is a compound of formula (II)

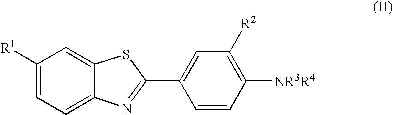

or a radiolabeled derivative, pharmaceutically acceptable salt, hydrate, solvate or prodrug of the compound, wherein:

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;

R is C₁-C₆ alkyl;

R² is hydrogen or halo;

R³ is hydrogen, C₁-C₆ alkyl, C₂-C₆ alkenyl or C₂-C₆ alkynyl; and

R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;

provided that when R¹ is hydrogen or —OH, R² is hydrogen and R⁴ is —¹¹CH₃, then R³ is C₂-C₆ alkyl, C₂-C₆ alkenyl or C₂-C₆ alkynyl; and

further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl.

In one embodiment, R² in the compounds of formula (II) contains a radioactive halo. Thus, for example, one compound of formula (II) for use in combination with any of the embodiments described herein is 2-(3-¹⁸F-Fluoro-4-methylamino-phenyl)-benzothiazol-6-ol:

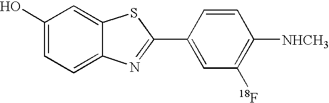

“Alkyl” refers to a saturated straight or branched chain hydrocarbon radical. Examples include without limitation methyl, ethyl, propyl, iso-propyl, butyl, iso-butyl, tert-butyl, n-pentyl and n-hexyl. The term “lower alkyl” refers to C₁-C₆ alkyl.

“Alkenyl” refers to an unsaturated straight or branched chain hydrocarbon radical comprising at least one carbon to carbon double bond. Examples include without limitation ethenyl, propenyl, iso-propenyl, butenyl, iso-butenyl, tert-butenyl, n-pentenyl and n-hexenyl.

“Alkynyl” refers to an unsaturated straight or branched chain hydrocarbon radical comprising at least one carbon to carbon triple bond. Examples include without limitation ethynyl, propynyl, iso-propynyl, butynyl, iso-butynyl, tert-butynyl, pentynyl and hexynyl.

“Alkoxy” refers to an alkyl group bonded through an oxygen linkage.

“Halo” refers to a fluoro, chloro, bromo or iodo radical.

“Radioactive halo” refers to a radioactive halo, i.e. radiofluoro, radiochloro, radiobromo or radioiodo.

In another embodiment, the thioflavin compound of formula (I) is selected from the group consisting of structures 1-45 or a radiolabeled derivative thereof:

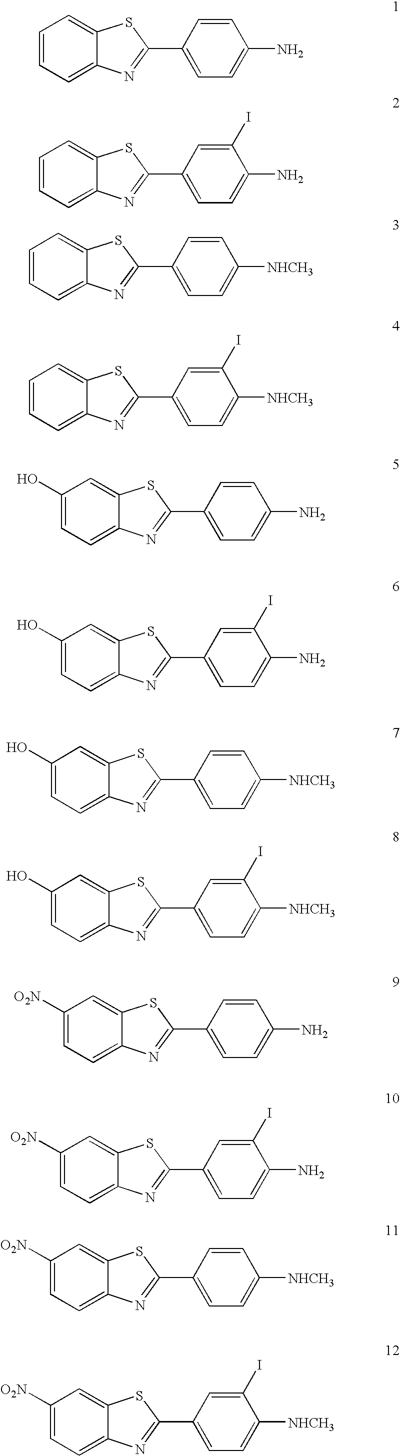

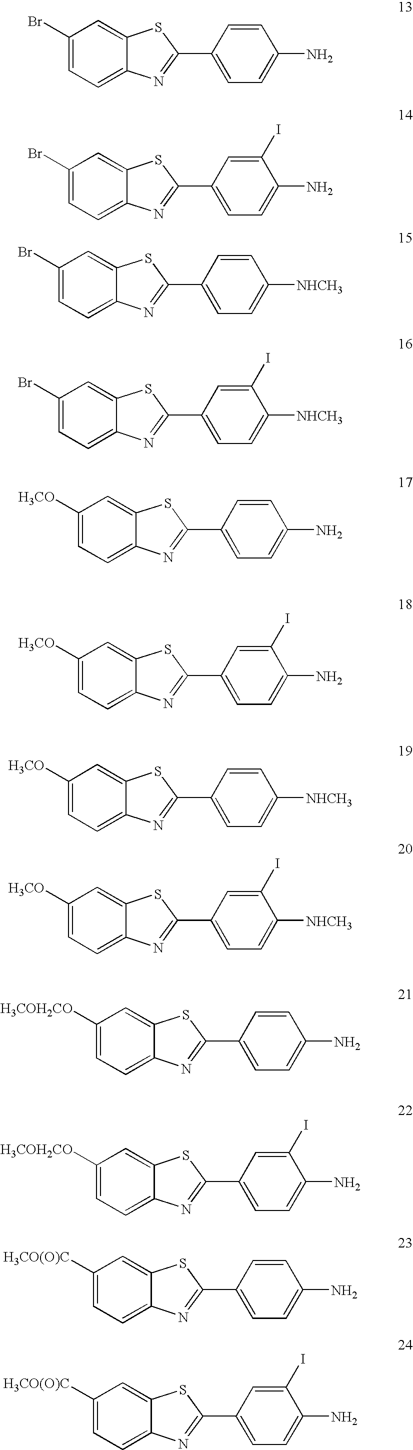

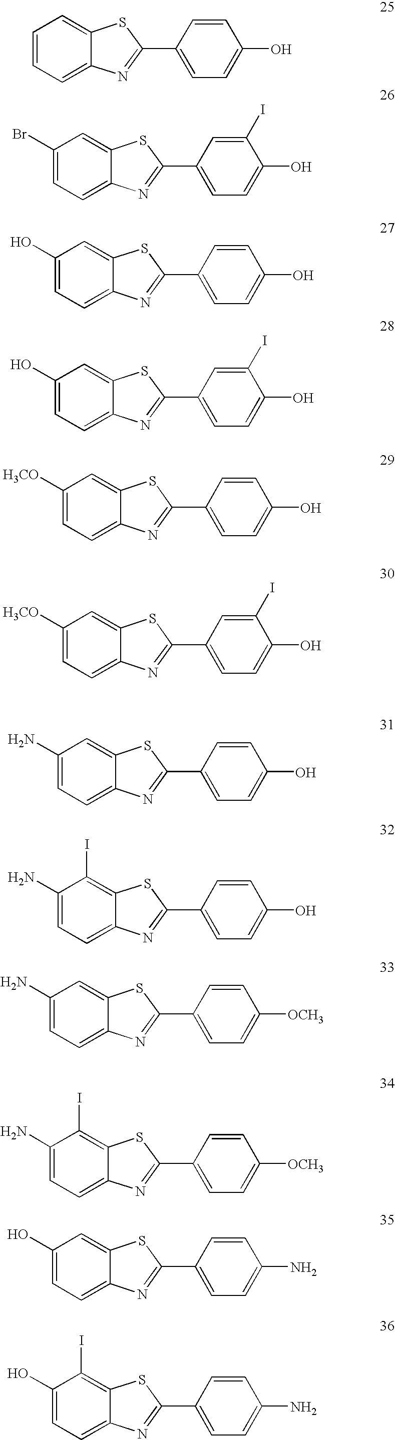

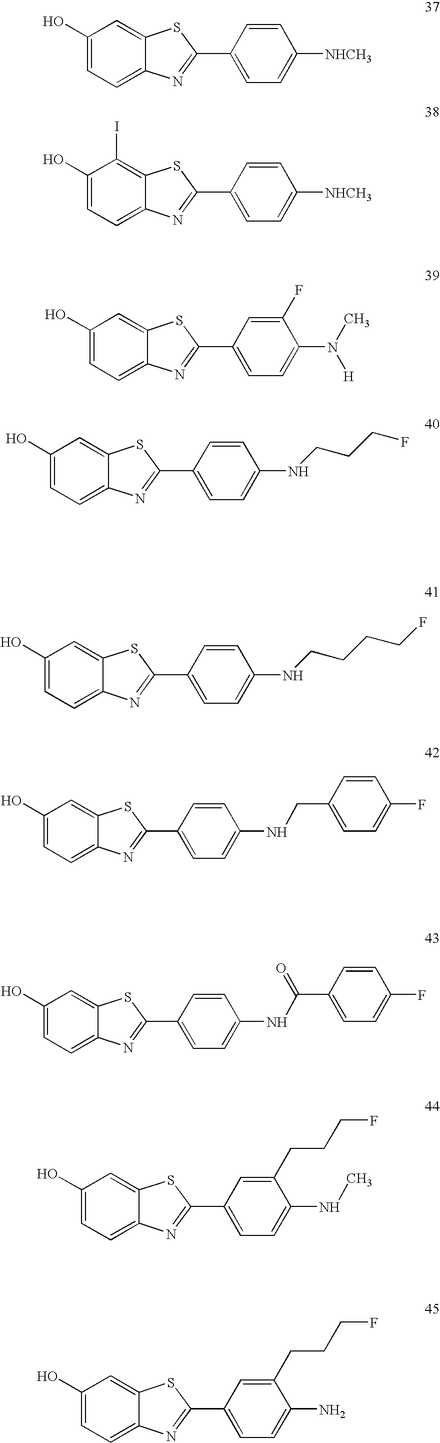

In the compounds I-45, at least one of the substituent moieties comprises a detectable label as defined above.

In preferred embodiments, the amyloid imaging agent is {N-methyl-¹¹C}2-[4′-(methylamino)phenyl]6-hydroxybenzothiazole (“[¹¹C]PIB”) or {N-methyl-³H}2-[4′-(methylamino)phenyl]6-hydroxybenzothiazole (“[³H]PIB”).

“Effective amount” refers to the amount required to produce a desired effect. Examples of an “effective amount” include amounts that enable detecting and imaging of amyloid deposit(s) in vivo or in vitro, that yield acceptable toxicity and bioavailability levels for pharmaceutical use, and/or prevent cell degeneration and toxicity associated with fibril formation.

Compounds of formulas (I) and (II) or structures 1-45, also referred to herein as “thioflavin compounds,” “thioflavin derivatives,” or “amyloid imaging agents,” have each of the following characteristics: (1) specific binding to synthetic AP in vitro and (2) ability to cross a non-compromised blood brain barrier in vivo.

The thioflavin compounds and radiolabeled derivatives thereof of formulas (I) (II) and structures 1-45 cross the blood brain barrier in vivo and bind to AP deposited in neuritic (but not diffuse) plaques, to AP deposited in cerebrovascular amyloid, and to the amyloid consisting of the protein deposited in NFT. The present compounds are non-quaternary amine derivatives of Thioflavin S and T which are known to stain amyloid in tissue sections and bind to synthetic AP in vitro. Kelenyi J. Histochem. Cytochem. 15: 172 (1967); Burns et al. J Path. Bact. 94:337 (1967); Guntern et al. Experientia 48: 8 (1992); LeVine Meth. Enzymol. 309: 274 (1999).

The method of this invention determines the presence and location of amyloid deposits in an organ or body area, preferably brain, of a patient. The present method comprises administration of a detectable quantity of an amyloid imaging agent of formulas (I) or (II). In some embodiments, the amyloid imaging agent is chosen from structures 1-45, as shown above. An amyloid imaging agent may be administered to a patient as a pharmaceutical composition or a pharmaceutically acceptable water-soluble salt thereof.

“Pharmaceutically acceptable salt” refers to an acid or base salt of the inventive compound, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

Generally, the dosage of the detectably labeled thioflavin derivative will vary depending on considerations such as age, condition, sex, and extent of disease in the patient, contraindications, if any, concomitant therapies and other variables, to be adjusted by a physician skilled in the art. Dosage can vary from 0.001 μg/kg to 10 μg/kg, preferably 0.01 μg/kg to 1.0 μg/kg.

Administration to the subject may be local or systemic and accomplished intravenously, intraarterially, intrathecally (via the spinal fluid) or the like. Administration may also be intradermal or intracavitary, depending upon the body site under examination. After a sufficient time has elapsed for the compound to bind with the amyloid, for example 30 minutes to 48 hours, the area of the subject under investigation is examined by routine imaging techniques such as MRS/MRI, SPECT, planar scintillation imaging, PET, and any emerging imaging techniques, as well. The exact protocol will necessarily vary depending upon factors specific to the patient, as noted above, and depending upon the body site under examination, method of administration and type of label used; the determination of specific procedures would be routine to the skilled artisan. For brain imaging, preferably, the amount (total or specific binding) of the bound radioactively labeled thioflavin derivative or analogue of the present invention is measured and compared (as a ratio) with the amount of labeled thioflavin derivative bound to the cerebellum of the patient. This ratio is then compared to the same ratio in age-matched normal brain.

The amyloid imaging agents of the present invention are advantageously administered in the form of injectable compositions, but may also be formulated into well known drug delivery systems (e.g., oral, rectal, parenteral (intravenous, intramuscular, or subcutaneous), intracisternal, intravaginal, intraperitoneal, local (powders, ointments or drops), or as a buccal or nasal spray). A typical composition for such purpose comprises a pharmaceutically acceptable carrier. For instance, the composition may contain about 10 mg of human serum albumin and from about 0.5 to 500 micrograms of the labeled thioflavin derivative per milliliter of phosphate buffer containing NaCl. Other pharmaceutically acceptable carriers include aqueous solutions, non-toxic excipients, including salts, preservatives, buffers and the like, as described, for instance, in REMINGTON'S PHARMACEUTICAL SCIENCES, 15th Ed. Easton: Mack Publishing Co. pp. 1405-1412 and 1461-1487 (1975) and THE NATIONAL FORMULARY XIV., 14th Ed. Washington: American Pharmaceutical Association (1975), the contents of which are hereby incorporated by reference.

Particularly preferred amyloid imaging agents of the present invention are those that, in addition to specifically binding amyloid in vivo and capable of crossing the blood brain barrier, are also non-toxic at appropriate dosage levels and have a satisfactory duration of effect.

According to the present invention, a pharmaceutical composition comprising an amyloid imaging agent of formula (I) or (II) or structures 1-45, is administered to subjects in whom amyloid or amyloid fibril formation are anticipated, e.g., patients clinically diagnosed with Alzheimer's disease.

Examples of non-aqueous solvents are propylene glycol, polyethylene glycol, vegetable oil and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial, anti-oxidants, chelating agents and inert gases. The pH and exact concentration of the various components the pharmaceutical composition are adjusted according to routine skills in the art. See, Goodman and Gilman's THE PHARMACOLOGICAL BASIS FOR THERAPEUTICS (7th Ed.).

### Imaging

The invention employs amyloid imaging agents which, in conjunction with non-invasive neuroimaging techniques such as magnetic resonance spectroscopy (MRS) or imaging (MRI), or gamma imaging such as positron emission tomography (PET) or single-photon emission computed tomography (SPECT), are used to quantify amyloid deposition in vivo. These imaging techniques acquire data on many brain regions. Quantitation on specific regions is achieved by delineating “regions of interest or ROI”. The method involves imaging a patient to establish amyloid deposition.

The term “in vivo imaging” refers to any method which permits the detection of a labeled thioflavin derivative of formulas (I) or (II) or structures 1-45. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound. A “subject” is a mammal, preferably a human, and most preferably a human suspected of having a disease associated with amyloid deposition, such as AD and/or dementia. The term “subject” and “patient” are used interchangeably herein.

For purposes of in vivo imaging, the type of detection instrument available is a major factor in selecting a given label. For instance, radioactive isotopes and ¹⁸F are well suited for in vivo imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen radiolabel will lack a particulate emission, but will produce a large number of photons in a 140-200 keV range. For PET detection, the radiolabel will be a positron-emitting radionuclide such as ¹⁹F which will annihilate to form two 511 keV gamma rays which will be detected by the PET camera.

In the present invention, amyloid binding compounds, which are useful for in vivo imaging and quantification of amyloid deposition, are administered to a patient. These compounds are to be used in conjunction with non-invasive neuroimaging techniques such as magnetic resonance spectroscopy (MRS) or imaging (MRI), positron emission tomography (PET), and single-photon emission computed tomography (SPECT). In accordance with this invention, the thioflavin derivatives may be labeled with ¹⁹F or ¹³C for MRS/MRI by general organic chemistry techniques known to the art. See, e.g., March, J. ADVANCED ORGANIC CHEMISTRY: REACTIONS, MECHANISMS, AND STRUCTURE (3rd Edition, 1985), the contents of which are hereby incorporated by reference. The thioflavin derivatives also may be radiolabeled with ¹⁸F, ¹¹C, ⁷⁵Br, or ⁷⁶Br for PET by techniques well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The thioflavin derivatives also may be radiolabeled with 1231 for SPECT by any of several techniques known to the art. See, e.g., Kulkami, Int. J. Rad. Appl. & Inst. (Part B) 18: 647 (1991), the contents of which are hereby incorporated by reference. In addition, the thioflavin derivatives may be labeled with any suitable radioactive iodine isotope, such as, but not limited to ¹³¹I, ¹²⁵I, or ¹²³I, by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art. See, Satyamurthy and Barrio J. Org. Chem. 48: 4394 (1983), Goodman et al., J. Org. Chem. 49: 2322 (1984), and Mathis et al., J Labell. Comp. and Radiopharm. 1994: 905; Chumpradit et al., J. Med. Chem. 34: 877 (1991); Zhuang et al., J. Med. Chem. 37: 1406 (1994); Chumpradit et al., J. Med. Chem. 37: 4245 (1994). For example, a stable triazene or tri-alkyl tin derivative of thioflavin or its analogues is reacted with a halogenating agent containing ¹³¹I, ¹²⁵I, ¹²³I, ⁷⁶Br, ⁷⁵Br, ¹⁸F or ¹⁹F. Thus, the stable tri-alkyl tin derivatives of thioflavin and its analogues are novel precursors useful for the synthesis of many of the radiolabeled compounds within the present invention. As such, these tri-alkyl tin derivatives are one embodiment of this invention.

The thioflavin derivatives also may be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled thioflavin derivative can then be used to detect amyloid deposits. Preparing radiolabeled derivatives of Tc^{99m} is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v) O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

The methods of the present invention may use isotopes detectable by nuclear magnetic resonance spectroscopy for purposes of in vivo imaging and spectroscopy. Elements particularly useful in magnetic resonance spectroscopy include ¹⁹F and ¹³C.

Suitable radioisotopes for purposes of this invention include beta-emitters, gamma-emitters, positron-emitters, and x-ray emitters. These radioisotopes include ¹³¹I, ¹²³I, ¹⁸F, ¹¹C, ⁷⁵Br, and ⁷⁶Br. Suitable stable isotopes for use in Magnetic Resonance Imaging (MRI) or Spectroscopy (MRS), according to this invention, include ¹⁹F and ¹³C. Suitable radioisotopes for in vitro quantification of amyloid in homogenates of biopsy or post-mortem tissue include ¹²⁵I, ¹⁴C, and ³H. The preferred radiolabels are ¹¹C or ¹⁸F for use in PET in vivo imaging, ¹²³I for use in SPECT imaging, ¹⁹F for MRS/MRI, and ³H or ¹⁴C for in vitro studies. However, any conventional method for visualizing imaging agents can be utilized in accordance with this invention.

The ability of the compound of formulas (I) and (II) or structures 1-45 to specifically bind to amyloid plaques over neurofibrially tangles is particularly true at concentrations less than 10 nM, which includes the in vivo concentration range of PET radiotraces. At these low concentrations, in homogenates of brain tissue which contain only tangles and no plaques, significant binding does not result when compared to control brain tissue containing neither plaques nor tangles. However, incubation of homogenates of brain tissue which contains mainly plaques and some tangles with radiolabeled compounds of formulas (I) and (II) or structures 1-45, results in a significant increase in binding when compared to control tissue without plaques or tangles. This data suggests the advantage that these compounds are specific for Aβ deposits at concentrations less than 10 nM. These low concentrations are then detectable with PET studies, making PET detection using radiolabeled compounds of formulas (I) and (II) or structures 1-45 which are specific for Aβ deposits possible. The use of such compounds permits PET detection in Aβ deposits such as those found in plaques and cerebrovascular amyloid. Since it has been reported that levels of insoluble, deposited Aβ in the frontal cortex are increased prior to tangle formation, this would suggest that radiolabeled compounds of formulas (I) and (II) or structures 1-45, used as PET tracers, would be specific for the earliest changes in AD cortex. Naslund et al. JAMA 283:1571 (2000).

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

The following examples are given to illustrate the present invention. It should be understood, however, that the invention is not to be limited to the specific conditions or details described in these examples. Throughout the specification, any and all references to a publicly available document, including U.S. patents, are specifically incorporated into this patent application by reference.

### Data Analysis of Amyloid Imaging

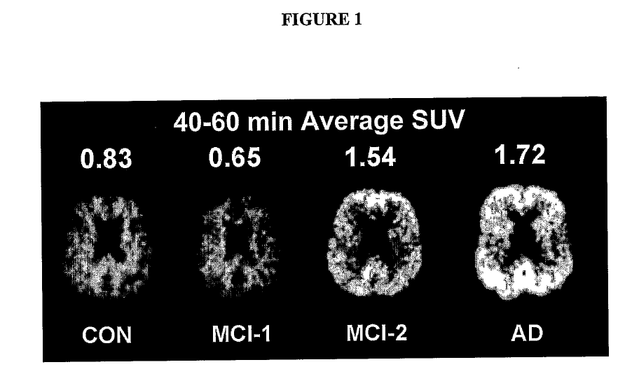

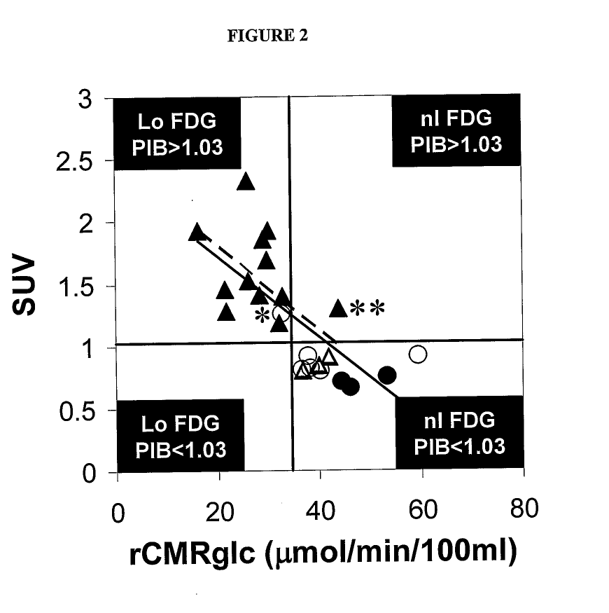

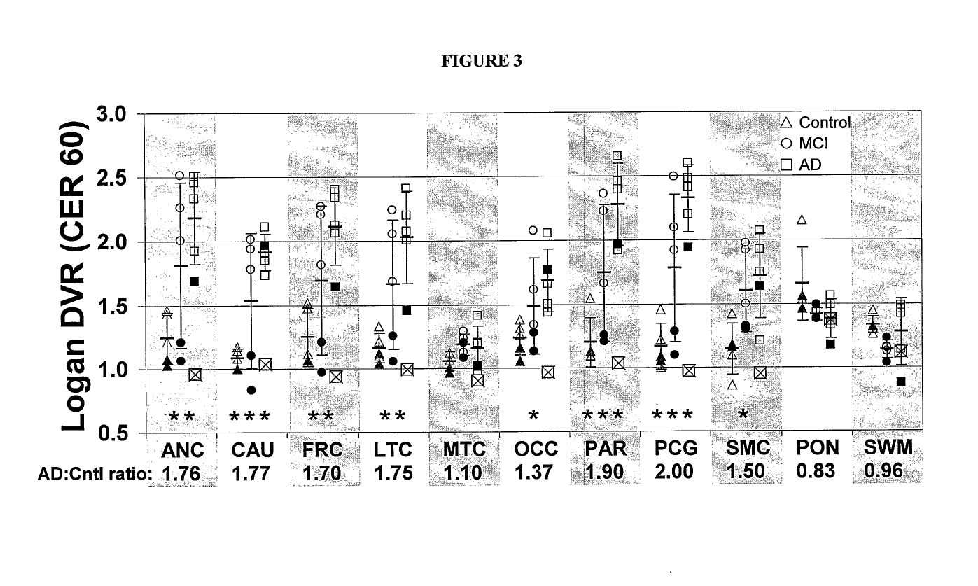

The data obtained can be quantitatively expressed in terms of Standardized Uptake Value (SUV) or in terms of pharmacokinetic modeling parameters such as the Logan distribution volume ratio (DVR) to a reference tissue such as cerebellum. Subjects who are more than one standard deviation above the typical control value of SUV or DVR would be considered to have a “positive” test and be considered to be prodromal to a clinical diagnosis of an amyloid deposition disease such as AD. Specifically, subjects will be considered “positive” if their 40-60 min average SUV is greater than 1.0 in frontal, parietal or posterior cingulate cortex. This value clearly separated AD patients from controls in the initial human study (Klunk, et al., 2004, Ann. Neurol., 55(3):306-19) (see FIG. 2). Likewise, subjects can be considered “positive” if their Logan DVR value exceeds 1.5 in frontal, parietal or posterior cingulate cortex (see FIG. 3). These brain areas and exact cutoffs are given only as examples and further work may disclose additional brain areas that are useful and the cutoff values may be refined and other modeling techniques (such as compartmental modeling, graphical analysis, reference tissue modeling or spectral analysis) may be applied to determine the cutoffs. In addition, the scan data can be qualitatively interpreted from images such as those in FIG. 1 that reflect the regional brain distribution of either SUV, Logan DVR or other parameters in which one having ordinary skill in the art of interpreting PET scans can determine that the qualitative amount and distribution of amyloid is consistent with a prodromal phase of a clinically diagnosed amyloid deposition disease.

### SYNTHESIS EXAMPLES

Compounds of formulas (I) and (II), and the formulae of structures 1-45, can be prepared by methods that are well known in the art. See, e.g., WO 02/16333 and U.S. Patent Publication No. 2003/0236391, published Dec. 25, 2003, the entire contents of which are herein incorporated by reference.

All of the reagents used in the synthesis were purchased from Aldrich Chemical Company and used without further purification, unless otherwise indicated. Melting points were determined on MeI-TEMP II and were uncorrected. The ¹H NMR spectra of all compounds were measured on Bruker 300 using TMS as internal reference and were in agreement with the assigned structures. The TLC was performed using Silica Gel 60 F₂₅₄ from EM Sciences and detected under UV lamp.

Flash chromatography was performed on silica gel 60 (230-400 mesh. Purchased from Mallinckrodt Company. The reverse phase TLC were purchased from Whiteman Company.

### General Methodology for Synthesis of Compound of Formula (I):

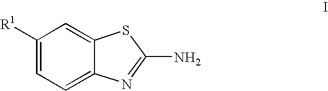

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo, wherein one or more of the atoms of R¹ may be a radiolabeled atom;

R is C₁-C₆ alkyl, wherein one or more of the carbon atoms may be a radiolabeled atom;

is hydrolysed by one of the following two procedures:

### Preparation of 2-Aminothiophenol Via Hydrolysis

The 6-substituted 2-aminobenzothiazole (172 mmol) is suspended in 50% KOH (180 g KOH dissolved in 180 mL water) and ethylene glycol (40 mL). The suspension is heated to reflux for 48 hours. Upon cooling to room temperature, toluene (300 mL) is added and the reaction mixture is neutralized with acetic acid (180 mL). The organic layer is separated and the aqueous layer is extracted with another 200 mL of toluene. The toluene layers are combined and washed with water and dried over MgSO₄. Evaporation of the solvent gives the desired product.

### Preparation of 2-Aminothiophenol Via Hydrazinolysis

The 6-substituted-benzothiazole (6.7 mmol) is suspended in ethanol (11 mL, anhydrous) and hydrazine (2.4 mL) is added under a nitrogen atmosphere at room temperature. The reaction mixture is heated to reflux for 1 hour. The solvent is evaporated and the residue is dissolved into water (10 mL) and adjusted to a pH of 5 with acetic acid. The precipitate is collected with filtration and washed with water to give the desired product.

The resulting 5-substituted-2-amino-1-thiophenol of the form

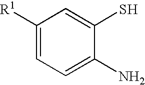

is coupled to a benzoic acid of the form:

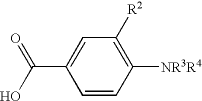

wherein R² is hydrogen, and R³ and R⁴ are independently hydrogen, C₁-C₆ alkyl, C₂-C₆ alkenyl or C₂-C₆ alkynyl

by the following methodology:

A mixture of the 5-substituted 2-aminothiophenol (4.0 mmol), the benzoic acid (4.0 mmol), and polyphosphoric acid (PPA) (10 g) is heated to 220° C. for 4 hours. The reaction mixture is cooled to room temperature and poured into 10% potassium carbonate solution (˜400 mL). The precipitate is collected by filtration under reduced pressure to give the desired product, which can be purified by flash chromatography or recrystallization.

The R² hydrogen can be substituted with either a non-radioactive halo or a radioactive halo by the following reaction:

To a solution of 6-substituted 2-(4′-aminophenyl)-benzothiazole (1 mg) in 250 μL acetic acid in a sealed vial is added 40 μL of chloramine-T solution (28 mg dissolved in 500 μL acetic acid) followed by 27 μL (ca. 5 mCi) of sodium [¹²⁵I]iodide (specific activity 2,175 Ci/mmol). The reaction mixture is stirred at room temperature for 2.5 hours and quenched with saturated sodium hydrogensulfite solution. After dilution with 20 ml of water, the reaction mixture is loaded onto C8 Plus SepPak and eluted with 2 ml methanol. Depending on the nature of the substituent on the 6-position, protecting groups may need to be employed. For example, the 6-hydroxy group is protected as the methanesulfonyl(mesyloxy) derivative. For deprotection of the methanesulfonyl group, 0.5 ml of 1 M NaOH is added to the eluted solution of radioiodinated intermediate. The mixture is heated at 50° C. for 2 hours. After being quenched by 500 μL of 1 M acetic acid, the reaction mixture is diluted with 40 mL of water and loaded onto a C8 Plus SepPak. The radioiodinated product, having a radioactivity of ca. 3 mCi, is eluted off the SepPak with 2 mL of methanol. The solution is condensed by a nitrogen stream to 300 μL and the crude product is purified by HPLC on a Phenomenex ODS column (MeCN/TEA buffer, 35:65, pH 7.5, flow rate 0.5 mL/minute up to 4 minutes, 1.0 mL/minute at 4-6 minutes, and 2.0 mL/minute after 6 minutes, retention time 23.6). The collected fractions are loaded onto a C8 Plus SepPak. Elution with 1 mL of ethanol gave ca. 1 mCi of the final radioiodinated product.

When either or both R³ and R⁴ are hydrogen, then R³ and R⁴ can be converted to C₁-C₆ alkyl, C₂-C₆ alkenyl or C₂-C₆ alkynyl by reaction with an alkyl, alkenyl or alkynyl halide under the following conditions:

For dialkylation: To a solution of 6-substituted 2-(4′-aminophenyl)-benzothiazole (0.59 mmol) in DMSO (anhydrous, 2 ml) are added alkyl, alkenyl, or alkynyl halide (2.09 mmol), and K₂CO₃ (500 mg, 3.75 mmol). The reaction mixture is heated at 140° C. for 16 hours. Upon cooling to room temperature, the reaction mixture is poured into water and extracted with ethyl acetate (3×10 mL). The organic layers are combined and the solvent is evaporated. The residue is purified by flash column to give the desired 6-substituted dimethylaminophenyl)-benzothiazole.

For monoalkylation: To a solution of 6-substituted 2-(4′-aminophenyl)benzothiazole (0.013 mmol) in DMSO (anhydrous, 0.5 ml) is added alkyl, alkenyl, or alkynyl halide (0.027 mmol) and anhydrous K₂CO₃ (100 mg, 0.75 mmol). The reaction mixture is heated at 100° C. for 16 hours. Upon cooling to room temperature, the reaction mixture is directly purified by normal phase preparative TLC to give the desired 6-substituted-2-(4′-methylaminophenyl)-benzothiazole derivatives.

When R² is hydrogen or a non-radioactive halo, R⁴ is C₁-C₆ alkyl, C₂-C₆ alkenyl or C₂-C₆ alkynyl, wherein the alkyl, alkenyl or alkynyl comprises a radioactive carbon or is substituted with a radioactive halo, the compound can be synthesized by one of the following sequences:

### For Radioactive Carbon Incorporation:

Approximately 1 Ci of [¹¹C]carbon dioxide is produced using a CTI/Siemens RDS 112 negative ion cyclotron by irradiation of a nitrogen gas (14N₂) target containing 1% oxygen gas with a 40 μA beam current of 11 MeV protons for 60 minutes. [¹¹C]Carbon dioxide is converted to [¹¹C]methyl iodide by first reacting it with a saturated solution of lithium aluminum hydride in THF followed by the addition of hydriodic acid at reflux temperature to generate [¹¹C]methyl iodide. The [¹¹C]methyl iodide is carried in a stream of nitrogen gas to a reaction vial containing the precursor for radiolabeling. The precursor, 6-substituted 2-(4′-aminophenyl)-benzothiazole (˜3.7 μmoles), is dissolved in 400 μL of DMSO. Dry KOH (10 mg) is added, and the 3 mL V-vial is vortexed for 5 minutes. No-carrier-added [¹¹C]methyl iodide is bubbled through the solution at 30 mL/minute at room temperature. The reaction is heated for 5 minutes at 95° C. using an oil bath. The reaction product is purified by semi-preparative HPLC using a Prodigy ODS-Prep column eluted with 60% acetonitrile/40% triethylammonium phosphate buffer pH 7.2 (flow at 5 mL/minute for 0-7 minutes then increased to 15 mL/minute for 7-30 minutes). The fraction containing [N-methyl-¹¹C]6-substituted 2-(4′-methylaminophenyl)-benzothiazole (at about 15 min) is collected and diluted with 50 mL of water and eluted through a Waters C18 SepPak Plus cartridge. The C18 SepPak is washed with 10 mL of water, and the product is eluted with 1 mL of ethanol (absolute) into a sterile vial followed by 14 mL of saline. Radiochemical and chemical purities are >95% as determined by analytical HPLC (k′=4.4 using the Prodigy ODS(3) analytical column eluted with 65/35 acetonitrile/triethylammonium phosphate buffer pH 7.2). The radiochemical yield averages 17% at EOS based on [¹¹C]methyl iodide, and the specific activity averages about 160 GBq/μmol (4.3 Ci/μmol) at end of synthesis.

For radioactive Halogen Incorporation:

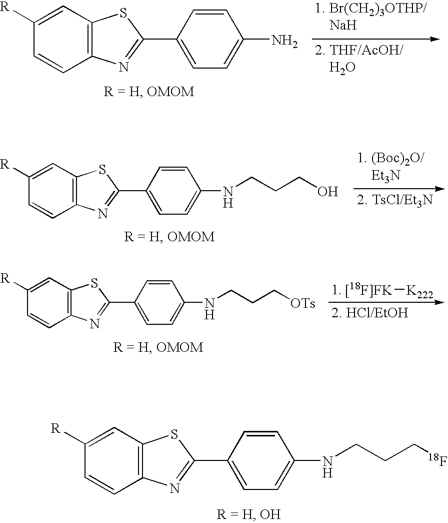

A mixture of 6-substituted 2-(4′-aminophenyl)-benzathiazole (protecting groups may be necessary depending on the nature of the 6-substituent as noted above) (0.22 mmol), NaH (4.2 mmol) and 2-(-3-bromopropoxy)tetrahydro-2-H-pyran (0.22 mmol) in THF (8 mL) is heated to reflux for 23 hours. The solvent is removed by distillation and the residue is dissolved in to ethyl acetate and water, the organic layer is separated and the aqueous layer is extracted with ethyl acetate (10 mL×6). The organic layer is combined and dried over MgSO₄ and evaporated to dryness. The residue is added AcOH/THF/H₂O solution (5 mL, 4/2/1) and heated to 10° C. for 4 hours. The solvent is removed by evaporation and the residue is dissolved in ethyl acetate (˜10 mL) washed by NaHCO₃ solution, dried over MgSO₄ and evaporated to dryness to give a residue which is purified with preparative TLC (hexane:ethyl acetate=60:40) to give the desired 6-substituted 2-(4′-(3″-hydroxypropylamino)-phenyl)-benzothiazole (45%).

To a solution of 6-substituted 2-(4′-(3″-hydroxypropylamino)-phenyl)-benzathiazole (0.052 mmol) and Et₃N (0.5 ml) dissolved in acetone (5 mL) is added (BOC)₂O (50 mg, 0.22 mmol). The reaction mixture is stirred at room temperature for 6 hours followed by addition of tosyl chloride (20 mg, 0.11 mmol). The reaction mixture is stirred at room temperature for another 24 hours. The solvent is removed and the residue is dissolved into ethyl acetate (10 mL), washed with NaCO₃ solution, dried over MgSO₄, evaporated, and purified with flash column (Hexane/ethyl acetate=4/1) to give the desired 6-substituted 2-(4′-(3″-toluenesulfonoxypropylamino)-phenyl)-benzothiazole (13%). This 6-substituted 2-(4′-(3″-toluenesulfonoxypropylamino)-phenyl)-benzothiazole is then radiofluorinated by standard methods as follows:

A cyclotron target containing 0.35 mL of 95% [O-18]-enriched water is irradiated with 11 MeV protons at 20 μA of beam current for 60 minutes, and the contents are transferred to a 5 mL reaction vial containing Kryptofix 222 (22.3 mg) and K₂CO₃ (7.9 mg) in acetonitrile (57 μL). The solution is evaporated to dryness three times at 110° C. under a stream of argon following the addition of 1 mL aliquots of acetonitrile. To the dried [F-18]fluoride is added 3 mg of 6-substituted 2-(4′-(3″-toluenesulfonoxypropylamino)-phenyl)-benzothiazole in 1 mL DMSO, and the reaction vial is sealed and heated to 85° C. for 30 minutes. To the reaction vial, 0.5 mL of MeOH/HCl (concentrated) (2/1 v/v) is added, and the vial is heated at 120° C. for 10 minutes. After heating, 0.3 mL of 2 M sodium acetate buffer is added to the reaction solution followed by purification by semi-prep HPLC using a Phenomenex Prodigy ODS-prep C18 column (10 μm 250×10 mm) eluted with 40% acetonitrile/60% 60 mM triethylamine-phosphate buffer (v/v) pH 7.2 at a flow rate of 5 mL/minute for 15 minutes, then the flow is increased to 8 mL/minute for the remainder of the separation. The product, [F-18]6-substituted 2-(4′-(3″-fluoropropylamino)-phenyl)-benzothiazole, is eluted at 20 minutes in a volume of about 16 mL. The fraction containing [F-18]6-substituted 2-(4′-(3″-fluoropropylamino)-phenyl)-benzothiazole is diluted with 50 mL of water and eluted through a Waters C18 SepPak Plus cartridge. The SepPak cartridge is then washed with 10 mL of water, and the product is eluted using 1 mL of ethanol (absol.) into a sterile vial. The solution is diluted with 10 mL of sterile normal saline for intravenous injection into animals. The [F-18]6-substituted 2-(4′-(3″-fluoropropylamino)-phenyl)-benzothiazole product is obtained in 2-12% radiochemical yield at the end of the 120 minute radiosynthesis (not decay corrected) with an average specific activity of 1500 Ci/mmol.

### Example 1

### [N-Methyl-¹¹C]-2-(4′-Dimethylaminophenyl)-6-methoxy-benzothiazole was synthesized according to Scheme I

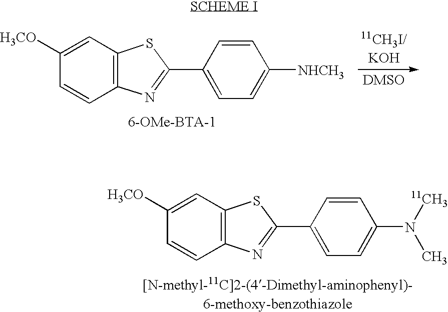

Approximately 1 Ci of [¹¹C]carbon dioxide was produced using a CTI/Siemens RDS 112 negative ion cyclotron by irradiation of a nitrogen gas (¹⁴N₂) target containing 1% oxygen gas with a 40 μA beam current of 11 MeV protons for 60 minutes. [¹¹C]Carbon dioxide is converted to [¹¹C]methyl iodide by first reacting it with a saturated solution of lithium aluminum hydride in THF followed by the addition of hydriodic acid at reflux temperature to generate [¹¹C]methyl iodide. The [¹¹C]methyl iodide is carried in stream of nitrogen gas to a reaction vial containing the precursor for radiolabeling. The precursor, 6-CH₃O-BTA-1 (1.0 mg, 3.7 μmoles), was dissolved in 400 μL of DMSO. Dry KOH (10 mg) was added, and the 3 mL V-vial was vortexed for 5 minutes. No-carrier-added [¹¹C]methyl iodide was bubbled through the solution at 30 mL/minute at room temperature. The reaction was heated for 5 minutes at 95° C. using an oil bath. The reaction product was purified by semi-preparative HPLC using a Prodigy ODS-Prep column eluted with 60% acetonitrile/40% triethylammonium phosphate buffer pH 7.2 (flow at 5 mL/minute for 0-7 minutes then increased to 15 mL/minute for 7-30 minutes). The fraction containing [N-Methyl-¹¹C]2-(4′-Dimethylaminophenyl)-6-methoxy-benzothiazole (at about 15 minutes) was collected and diluted with 50 mL of water and eluted through a Waters C18 SepPak Plus cartridge. The C18 SepPak was washed with 10 mL of water, and the product was eluted with 1 mL of ethanol (absolute) into a sterile vial followed by 14 mL of saline. Radiochemical and chemical purities were >95% as determined by analytical HPLC (k′=4.4 using the Prodigy ODS(3) analytical column eluted with 65/35 acetonitrile/triethylammonium phosphate buffer pH 7.2). The radiochemical yield averaged 17% at EOS based on [¹¹C]methyl iodide, and the specific activity averaged about 160 GBq/μmol (4.3 Ci/μmol) at end of synthesis.

### Example 2

### 2-(3′-¹²⁵I-iodo-4′-amino-phenyl)-benzothiazol-6-ol was synthesized according to Scheme II

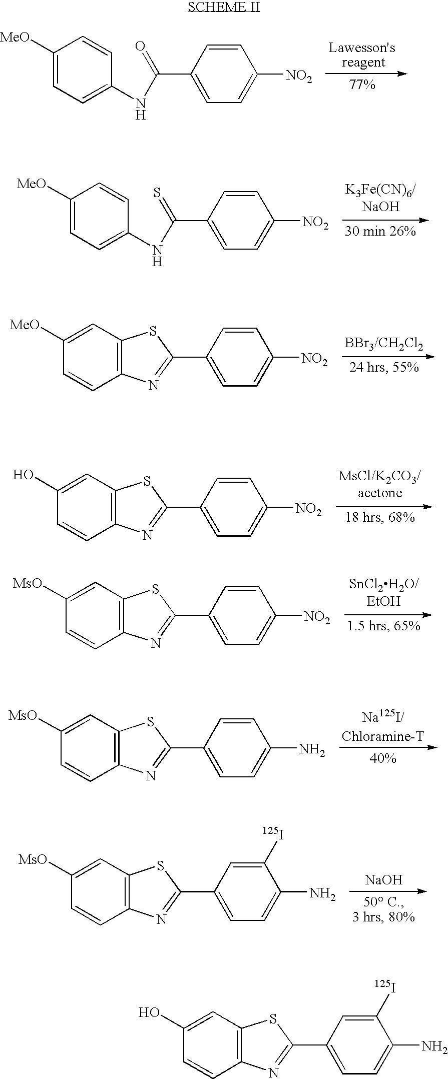

To a solution of 2-(4′-aminophenyl)-6-methanesulfonoxy-benzothiazole (1 mg) in 250 μL acetic acid in a sealed vial was added 40 μL of chloramine T solution (28 mg dissolved in 500 μL acetic acid) followed by 27 μL (ca. 5 mCi) of sodium [¹²⁵I]iodide (specific activity 2,175 Ci/mmol). The reaction mixture was stirred at room temperature for 2.5 hours and quenched with saturated sodium hydrogensulfite solution. After dilution with 20 ml of water, the reaction mixture was loaded onto C8 Plus SepPak and eluted with 2 ml methanol. For deprotection of the methanesulfonyl group, 0.5 ml of 1 M NaOH was added to the eluted solution of radioiodinated intermediate. The mixture was heated at 50° C. for 2 hours. After being quenched by 500 μL of 1 M acetic acid, the reaction mixture was diluted with 40 mL of water and loaded onto a C8 Plus SepPak. The radioiodinated product, having a radioactivity of ca. 3 mCi, was eluted off the SepPak with 2 mL of methanol. The solution was condensed by a nitrogen stream to 300 μL and the crude product was purified by HPLC on a Phenomenex ODS column (MeCN/TEA buffer, 35:65, pH 7.5, flow rate 0.5 mL/minute up to 4 minutes, 1.0 mL/minute at 4-6 minutes, and 2.0 mL/minute after 6 minutes, retention time 23.6). The collected fractions were loaded onto a C8 Plus SepPak. Elution with 1 mL of ethanol gave ca. 1 mCi of the final radioiodinated product.

Preparation of the ¹²³I radiolabeled derivatives, proceeds similarly to the synthesis outlined above. For example, replacing sodium [¹²⁵I]iodide with sodium [¹²³I]iodide in the synthetic method would provide the 1231 radiolabeled compound. Such substitution of one radiohalo atom for another is well known in the art, see for example, Mathis C A, Taylor S E, Biegon A, Enas J D. [¹²⁵I]5-Iodo-6-nitroquipazine: a potent and selective ligand for the 5-hydroxytryptamine uptake complex I. In vitro studies. Brain Research 1993; 619:229-235; Jagust W, Eberling J L, Roberts J A, Brennan K M, Hanrahan S M, Van Brocklin H, Biegon A, Mathis C A. In vivo imaging of the 5-hydroxytryptamine reuptake site in primate brain using SPECT and [¹²³I]5-iodo-6-nitroquipazine. European Journal of Pharmacology 1993; 242:189-193; Jagust W J, Eberling J L, Biegon A, Taylor S E, VanBrocklin H, Jordan S, Hanrahan S M, Roberts J A, Brennan K M, Mathis C A. [Iodine-123]5-Iodo-6-Nitroquipazine: SPECT Radiotracer to Image the Serotonin Transporter. Journal of Nuclear Medicine 1996; 37:1207-1214.)

### Example 3

### 2-(3-¹⁸F-Fluoro-4-methylamino-phenyl)-benzothiazol-6-ol was synthesized according to Scheme III

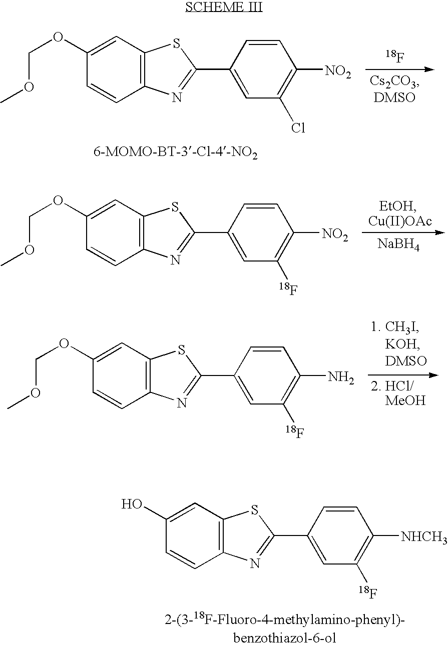

A cyclotron target containing 0.35 mL of 95% [0-18]-enriched water was irradiated with 11 MeV protons at 20 μA of beam current for 60 minutes, and the contents were transferred to a 5 mL reaction vial containing 2 mg Cs₂CO₃ in acetonitrile (57 μL). The solution was evaporated to dryness at 110° C. under a stream of argon three times using 1 mL aliquots of acetonitrile. To the dried [F-18]fluoride was added 6 mg of 6-MOMO-BT-3′-Cl-4′-NO₂ in 1 mL DMSO, and the reaction vial was sealed and heated to 120° C. for 20 minutes (radiochemical incorporation for this first radiosynthesis step was about 20% of solubilized [F-18]fluoride). To the crude reaction mixture was added 8 mL of water and 6 mL of diethyl ether, the mixture was shaken and allowed to separate. The ether phase was removed and evaporated to dryness under a stream of argon at 120° C. To the dried sample, 0.5 mL of absolute EtOH was added along with 3 mg copper (II) acetate and 8 mg of NaBH₄. The reduction reaction was allowed to proceed for 10 minutes at room temperature (the crude yield for the reduction step was about 40%). To the reaction mixture was added 8 mL of water and 6 mL of diethyl ether, the mixture was shaken and the ether phase separated. The diethyl ether phase was dried under a stream of argon at 120° C. To the reaction vial, 700 uL of DMSO was added containing 30 micromoles of CH₃I and 20 mg of dry KOH. The reaction vial was heated at 120° C. for 10 minutes. A solution of 700 uL of 2:1 MeOH/HCl (concentrated) was added and heated for 15 minutes at 120° C. After heating, 1 mL of 2 M sodium acetate buffer was added to the reaction solution followed by purification by semi-prep HPLC using a Phenomenex Prodigy ODS-prep C18 column (10 μm 250×10 mm) eluted with 35% acetonitrile/65% 60 mM triethylamine-phosphate buffer (v/v) pH 7.2 at a flow rate of 5 mL/minute for 2 minutes, then the flow was increased to 15 mL/minute for the remainder of the separation. The product, 2-(3-¹⁸F-fluoro-4-methylamino-phenyl)-benzothiazol-6-ol, eluted at ˜15 minutes in a volume of about 16 mL. The fraction containing 2-(3-18F-fluoro-4-methylamino-phenyl)-benzothiazol-6-ol was diluted with 50 mL of water and eluted through a Waters C18 SepPak Plus cartridge. The SepPak cartridge was then washed with 10 mL of water, and the product was eluted using 1 mL of ethanol (absol.) into a sterile vial. The solution was diluted with 10 mL of sterile normal saline for intravenous injection into animals. The 2-(3⁻¹⁸F-fluoro-4-methylamino-phenyl)-benzothiazol-6-ol product was obtained in 0.5% (n=4) radiochemical yield at the end of the 120 minute radiosynthesis (not decay corrected) with an average specific activity of 1000 Ci/mmol. The radiochemical and chemical purities of 2-(3-¹⁸F-fluoro-4-methylamino-phenyl)-benzothiazol-6-ol were assessed by radio-HPLC with UV detection at 350 nm using a Phenomenex Prodigy ODS(3) C18 column (5 μm, 250×4.6 mm) eluted with 40% acetonitrile/60% 60 mM triethylamine-phosphate buffer (v/v) pH 7.2. 2-(3-¹⁸F-Fluoro-4-methylamino-phenyl)-benzothiazol-6-ol had a retention time of 11 minutes at a flow rate of 2 mL/min (k′=5.5). The radiochemical purity was >99%, and the chemical purity was >90%. The radiochemical identity of 2-(3-¹⁸F-Fluoro-4-methylamino-phenyl)-benzothiazol-6-ol was confirmed by reverse phase radio-HPLC utilizing a quality control sample of the final radiochemical product co-injected with a authentic (cold) standard.

### Example 4

### 2-[4-(3⁻¹⁸F-Fluoro-propylamino)-phenyl]-benzothiazol-6-ol was synthesized according to Scheme IV

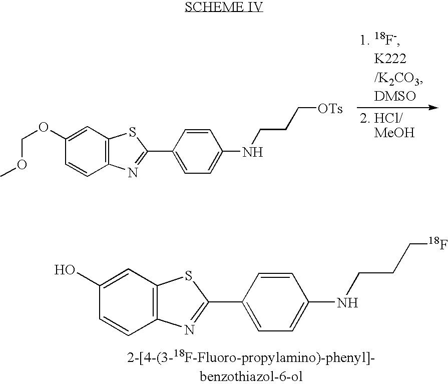

A cyclotron target containing 0.35 mL of 95% [0-18]-enriched water was irradiated with 11 MeV protons at 20 μA of beam current for 60 minutes, and the contents were transferred to a 5 mL reaction vial containing Kryptofix 222 (22.3 mg) and K₂CO₃ (7.9 mg) in acetonitrile (57 μL). The solution was evaporated to dryness three times at 110° C. under a stream of argon following the addition of 1 mL aliquots of acetonitrile. To the dried [F-18]fluoride was added 3 mg of 6-MOMO-BTA-N—Pr—Ots in 1 mL DMSO, and the reaction vial was sealed and heated to 85° C. for 30 minutes. To the reaction vial, 0.5 mL of MeOH/HCl (concentrated) (2/1 v/v) was added, and the vial was heated at 120° C. for 10 minutes. After heating, 0.3 mL of 2 M sodium acetate buffer was added to the reaction solution followed by purification by semi-prep HPLC using a Phenomenex Prodigy ODS-prep C18 column (10 μm 250×10 mm) eluted with 40% acetonitrile/60% 60 mM triethylamine-phosphate buffer (v/v) pH 7.2 at a flow rate of 5 mL/minute for 15 minutes, then the flow was increased to 8 mL/minute for the remainder of the separation. The product, [F-18]6-HO-BTA-N-PrF, eluted at 20 minutes in a volume of about 16 mL. The fraction containing [F-18]6-HO-BTA-N-PrF was diluted with 50 mL of water and eluted through a Waters C18 SepPak Plus cartridge. The SepPak cartridge was then washed with 10 mL of water, and the product was eluted using 1 mL of ethanol (absol.) into a sterile vial. The solution was diluted with 10 mL of sterile normal saline for intravenous injection into animals. The [F-18]6-HO-BTA-N-PrF product was obtained in 8±4% (n=8) radiochemical yield at the end of the 120 minute radiosynthesis (not decay corrected) with an average specific activity of 1500 Ci/mmol. The radiochemical and chemical purities of [F-18]6-HO-BTA-N-PrF were assessed by radio-HPLC with UV detection at 350 nm using a Phenomenex Prodigy ODS(3) C18 column (5 μm, 250×4.6 mm) eluted with 40% acetonitrile/60% 60 mM triethylamine-phosphate buffer (v/v) pH 7.2. [F-18]6-HO-BTA-N-PrF had a retention time of ˜12 minutes at a flow rate of 2 mL/minute (k′=6.1). The radiochemical purity was >99%, and the chemical purity was >90%. The radiochemical identity of [F-18]6-HO-BTA-N-PrF was confirmed by reverse phase radio-HPLC utilizing a quality control sample of the final radiochemical product co-injected with a authentic (cold) standard.

### Example 5

### Synthesis of 2-(3′-iodo-4′-aminophenyl)-6-hydroxy benzothiazole

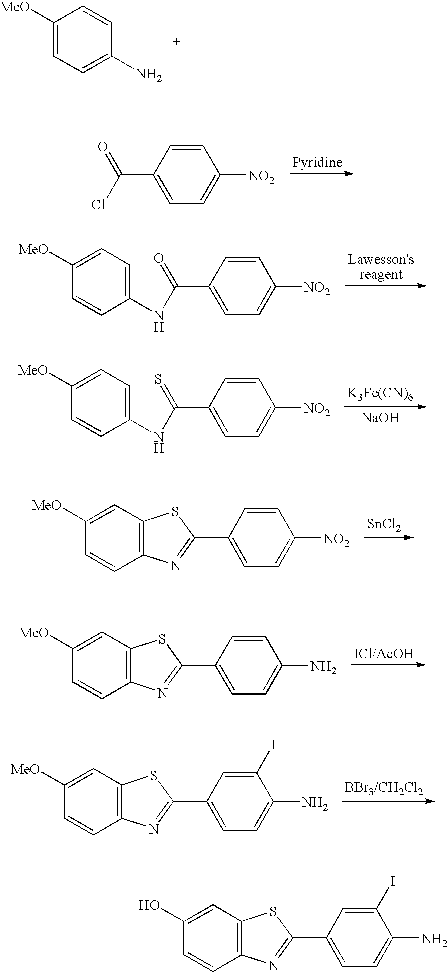

### Preparation of 4-Methoxy-4′-nitrobenzanilide

p-Anisidine (1.0 g, 8.1 mmol) was dissolved in anhydrous pyridine (15 ml), 4-nitrobenzoyl chloride (1.5 g, 8.1 mmol) was added. The reaction mixture was allowed to stand at room temperature for 16 hrs. The reaction mixture was poured into water and the precipitate was collected with filtrate under vacuum pressure and washed with 5% sodium bicarbonate (2×10 ml). The product was used in the next step without further purification. ¹HNMR (30 MHz, DMSO-d₆) δ: 10.46 (s, 1H, NH), 8.37 (d, J=5.5 Hz, 2H, H-3′,5′), 8.17 (d, J=6.3 Hz, 2H, H-2′,6′), 7.48 (d, J=6.6 Hz, 2H), 6.97 (d, J=6.5 Hz, 2H), 3.75 (s, 3H, MeO).

### Preparation of 4-Methoxy-4′-nitrothiobenzanilide

A mixture of 4-methoxy-4′-nitrothiobenzaniline (1.0 g, 3.7 mmol) and Lawesson's reagent (0.89 g, 2.2 mmol, 0.6 equiv.) in chlorobenzene (15 mL) was heated to reflux for 4 hrs. The solvent was evaporated and the residue was purified with flush column (hexane:ethyl acetate=4:1) to give 820 mg (77.4%) of the product as orange color solid. ¹HNMR (300 MHz, DMSO-d₆) δ: 8.29 (d, 2H, H-3′,5′), 8.00 (d, J=8.5 Hz, 2H, H-2′,6′), 7.76 (d, 2H), 7.03 (d, J=8.4 Hz, 2H), 3.808.37 (d, J=5.5 Hz, 2H, H-3′,5′), 8.17 (d, J=6.3 Hz, 2H, H-2′,6′), 7.48 (d, J=6.6 Hz, 2H), 6.97 (d, J=6.5 Hz, 2H), 3.75 (s, 3H, MeO). (s, 3H, MeO).

### Preparation of 6-Methoxy-2-(4-nitrophenyl)benzothiazole

4-Methoxy-4′-nitrothiobenzanilides (0.5 g, 1.74 mmol) was wetted with a little ethanol (˜0.5 mL), and 30% aqueous sodium hydroxide solution (556 mg 13.9 mmol. 8 equiv.) was added. The mixture was diluted with water to provide a final solution/suspension of 10% aqueous sodium hydroxide. Aliquots of this mixture were added at 1 min intervals to a stirred solution of potassium ferricyanide (2.29 g, 6.9 mmol, 4 equiv.) in water (5 mL) at 80-90° C. The reaction mixture was heated for a further 0.5 h and then allowed to cool. The participate was collected by filtration under vacuum pressure and washed with water, purified with flush column (hexane:ethyl acetate=4:1) to give 130 mg (26%) of the product. ¹HNMR (300 MHz, Acetone-d₆) δ: 8.45 (m, 4H), 8.07 (d, J=8.5 Hz, 1H, H-4), 7.69 (s, 1H, H-7), 7.22 (d, J=9.0 Hz, 1H, H-5), 3.90 (s, 3H, MeO)

### Preparation of 6-Methoxy-2-(4-aminophenyl)benzothiazole

A mixture of the 6-methoxy-2-(4-nitrophenyl)benzothiazoles (22 mg, 0.077 mmol) and tin(II) chloride (132 mg, 0.45 mmol) in boiling ethanol was stirred under nitrogen for 4 hrs. Ethanol was evaporated and the residue was dissolved in ethyl acetate (10 mL), washed with 1 N sodium hydroxide (2 mL) and water (5 mL), and dried over MgSO₄. Evaporation of the solvent gave 19 mg (97%) of the product as yellow solid.

### Preparation of 2-(3′-Iodo-4′-aminophenyl)-6-methoxybenzothiazole

To a solution of 2-(4′-aminophenyl)-6-methoxy benzothiazole (22 mg, 0.09 mmol) in glacial acetic acid (2.0 mL) was injected 1 M iodochloride solution in CH₂Cl₂ (0.10 mL, 0.10 mmol, 1.2 eq.) under N₂ atmosphere. The reaction mixture was stirred at room temperature for 16 hr. The glacial acetic acid was removed under reduced pressure and the residue was dissolved in CH₂Cl₂. After neutralizing the solution with NaHCO₃, the aqueous layer was separated and extracted with CH₂Cl₂. The organic layers were combined and dried over MgSO₄. Following the evaporation of the solvent, the residue was purified by preparative TLC(Hexanes:ethyl acetate=6:1) to give 2-(4′-amino-3′-iodophenyl)-6-methoxy benzothiazole (25 mg, 76%) as brown solid. ¹HNMR (300 MHz, CDCl₃) δ (ppm): 8.35 (d, J=2.0 Hz, 1H), 7.87 (dd, J₁=2.0 Hz, J₂=9.0 Hz, 1H), 7.31 (d, J=2.2 Hz, 1H), 7.04 (dd, J₁=2.2 Hz, J₂=9.0 Hz, 1H), 6.76 (d, J=9.0 Hz, 1H), 3.87 (s, 3H).

### Preparation of 2-(3′-Iodo-4′-aminophenyl)-6-hydroxybenzothiazole

To a solution of 2-(4′-Amino-3′-iodophenyl)-6-methoxy benzothiazole (5) (8.0 mg, 0.02 mmol) in CH₂Cl₂ (2.0 mL) was injected 1 M BBr₃ solution in CH₂Cl₂ (0.20 ml, 0.20 mmol) under N₂ atmosphere. The reaction mixture was stirred at room temperature for 18 hrs. After the reaction was quenched with water, the mixture was neutralized with NaHCO₃. The aqueous layer was extracted with ethyl acetate (3×3 mL). The organic layers were combined and dried over MgSO₄. The solvent was then evaporated under reduced pressure and the residue was purified by preparative TLC (Hexanes:ethyl acetate=7:3) to give 2-(3′-iodo-4′-aminophenyl)-6-hydroxybenzothiazole (4.5 mg, 58%) as a brown solid. ¹HNMR (300 MHz, acetone-d₆) δ (ppm): 8.69 (s, 1H), 8.34 (d, J=2.0 Hz, 1H), 7.77 (dd, J₁=2.0 Hz, J₂=8.4 Hz, 1H), 7.76 (d, J=8.8 Hz, 1H), 7.40 (d, J=2.4 Hz, 1H), 7.02 (dd, J₁=2.5 Hz, J₂=8.8 Hz, 1H), 6.94 (d, J=8.5 Hz, 1H), 5.47 (br., 2H). HRMS m/z 367.9483 (M+calcd for C₁₃H₉N₂OSI 367.9480).

### Example 6

### Synthesis of 2-(3′-iodo-4′-methylaminophenyl)-6-hydroxybenzothiazole

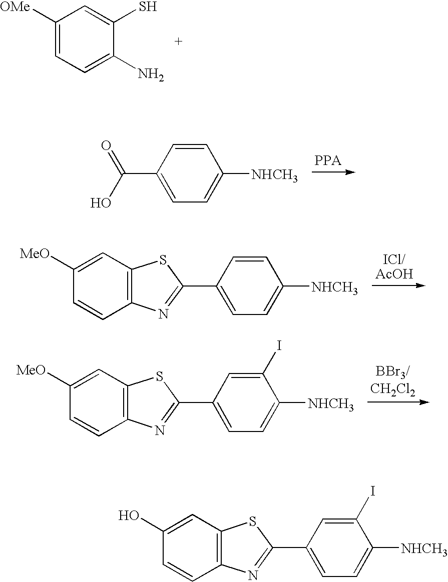

### Preparation of 6-Methoxy-2-(4-methylaminophenyl)benzothiazole

A mixture of 4-methylaminobenzoic acid (11.5 g, 76.2 mmol) and 5-methoxy-2-aminothiophenol (12.5, g, 80 mmol) was heated in PPA (˜30 g) to 170° C. under N₂ atmosphere for 1.5 hr. The reaction mixture was then cooled to room temperature and poured into 10% K₂CO₃ solution. The precipitate was filtered under reduced pressure. The crude product was re-crystallized twice from acetone/water and THF/water followed by the treatment with active with carbon to give 4.6 g (21%) of 6-Methoxy-2-(4-methylaminophenyl)benzothiazole as a yellow solid. ¹HNMR (300 MHz, acetone-d₆) δ: 7.84 (d, J=8.7 Hz, 2H, H-2′ 6′), 7.78 (dd, J₁=8.8 Hz, J₂=1.3 Hz, 1H, H-4), 7.52 (d, J=2.4 Hz, 1H, H-7), 7.05 (dd, J₁=8.8 Hz, J₂=2.4 Hz, H-5), 6.70 (d, J=7.6 Hz, 2H, H-3′ 5′), 5.62 (s, 1H, NH), 3.88 (s, 3H, OCH₃), 2.85 (d, J=6.2 Hz, 3H, NCH₃)

### Preparation of 2-(3′-Iodo-4′-methylaminophenyl)-6-methoxy benzothiazole

To a solution of 2-(4′-Methylaminophenyl)-6-methoxy benzothiazole (20 mg, 0.074 mmol) dissolved in glacial acetic acid (2 mL) was added Ic1 (90 μL, 0.15 mmol, 1.2 eq, 1M in CH₂Cl₂) under N₂. The reaction was allowed to stir at room temperature for 18 hr. The glacial acetic acid was then removed under reduced pressure. The residue was dissolved in CH₂Cl₂ and neutralized with NaHCO₃. The aqueous layer was extracted with CH₂Cl₂ and the organic layers were combined, dried over MgSO₄ and evaporated. The residue was purified with preparative TLC (Hexane: EA=2:1) to give 2-(4′-methylamino-3′-iodophenyl)-6-methoxy benzothiazole (8 mg, 27%) as brown solid. ¹HNMR (300 MHz, CDCl₃) δ (ppm): 8.39 (d, J=2.0 Hz, 1H), 7.88 (d, J=9.0 Hz, 1H), 7.33 (d, J=2.2 Hz, 1H), 7.06 (dd, J₁=2.2 Hz, J₂=9.0 Hz, 1H), 6.58 (d, J=9.0 Hz, 1H), 3.89 (s, 3H, OCH₃).

### Preparation of 2-(3′-Iodo-4′-methylamino-phenyl)-6-hydroxy benzothiazole

To a solution of 2-(4′-methylamino-3′-iodophenyl)-6-methoxy benzothiazole (12 mg, 0.03 mmol) dissolved in CH₂Cl₂(4 mL) was added BBr₃ (400 μl, 0.4 mmol, 1M in CH₂Cl₂) under N₂. The reaction was allowed to stir at room temperature for 18 hr. Water was then added to quench the reaction and the solution was neutralized with NaHCO₃, extracted with ethyl acetate (3×5 mL). The organic layers were combined, dried over MgSO₄ and evaporated. The residue was purified with preparative TLC (Hexane: EA=7:3) to give 2-(4′-methylamino-3′-iodophenyl)-6-hydroxy benzothiazole (5 mg, 43%) as brown solid. ¹HNMR (300 MHz, CDCl₃) δ (ppm): 8.37 (d, H=2.0 Hz, 1H), 7.88 (dd, J₁=2.0 Hz, J₂=8.4 Hz, 1H), 7.83 (d, J=8.8 Hz, 1H), 7.28 (d, J=2.4 Hz, 1H), 6.96 (dd, J₁=2.5 Hz, J₂=8.8 Hz, 1H), 6.58 (d, J=8.5 Hz, 1H), 2.96 (s, 3H, CH₃).

### Example 7

### Radiosynthesis of [¹²⁵I]6-OH-BTA-O-3′-I

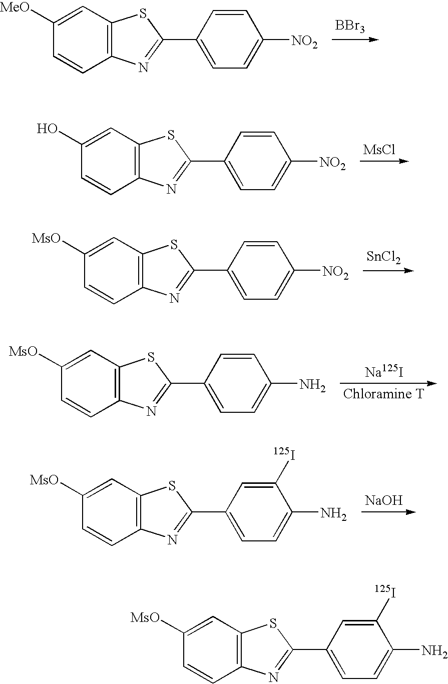

### Preparation of 2-(4′-Nitrophenyl)-6-hydroxybenzothiazole

To a suspension of 2-(4′-nitrophenyl)-6-methoxy benzothiazole (400 mg, 1.5 mmol) in CH₂Cl₂ (10 mL) was added BBr₃ (1M in CH₂Cl₂, 10 mL, 10 mmol). The reaction mixture was stirred at room temperature for 24 hr. The reaction was then quenched with water, and extracted with ethyl acetate (3×20 mL). The organic layers were combined and washed with water, dried over MgSO₄, and evaporated. The residue was purified by flash chromatography (silica gel, hexanes:ethyl acetate=1:1) to give the product as a yellow solid (210 mg, 55%). ¹HNMR (300 MHz, Acetone-d₆) δ (ppm): 9.02 (s, OH), 8.41 (d, J=9.1 Hz, 1H), 8.33 (d, J=9.1 Hz, 1H), 7.96 (d, J=8.6 Hz, 1H), 7.53 (d, J=2.4 Hz, 1H), 7.15 (dd, J₁=8.6 Hz, J₂=2.4 Hz, 1H).

### Preparation of 2-(4′-Nitrophenyl)-6-methylsulfoxy benzothiazole

To a solution of 2-(4′-nitrophenyl)-6-hydroxy benzothiazole (50 mg, 0.18 mmol) dissolved in acetone (7 mL, anhydrous) was added K₂CO₃ (100 mg, 0.72 mmol, powdered) and MsCl (200 ul). After stirring for 2 hrs, the reaction mixture was filtered. The filtrate was concentrated and the residue was purified by flash column (silica gel, hexane:ethyl acetate=4:1) to give 2-(4-nitrophenyl)-6-methylsulfoxy benzothiazole (44 mg, 68%) as pale yellow solid. ¹HNMR (300 MHz, acetone-d₆) δ (ppm): 8.50-8.40 (m, 4H), 8.29 (d, J=2.3 Hz, 1H), 8.23 (d, J=8.9 Hz, 1H), 7.61 (dd, J₁=2.3 Hz, J₂=8.9 Hz, 1H).

### Preparation of 2-(4′-Aminophenyl)-6-methylsulfoxy benzothiazole

To a solution of 2-(4′-nitrophenyl)-6-methylsulfoxy benzothiazole (35 mg, 0.10 mmol) dissolved in ethanol (10 mL) was added SnCl₂.2H₂O (50 mg). The reaction mixture was heated to reflux for 1.5 hr. The solvent was then removed under reduced pressure. The residue was dissolved in ethyl acetate (10 mL), washed with 1N NaOH, water, dried over MgSO₄. Evaporation of the solvent afforded 2-(4′-aminophenyl)-6-methylsulfoxy benzothiazole (21 mg, 65%) as pale brown solid. ¹HNMR (300 MHz, CDCl₃) δ (ppm): 8.02 (d, J=6.2 Hz, 1H), 7.92 (d, J=8.7 Hz, 2H), 7.84 (d, J=2.4 Hz, 1H), 7.38 (dd, J₁=2.4 Hz, J₂=6.2 Hz, 1H), 6.78 (d, J=8.7 Hz, 2H), 2.21 (s, 3H, CH₃).

### Example 8

### Radiosynthesis of [¹²⁵I]6-OH-BTA-1-3′-I

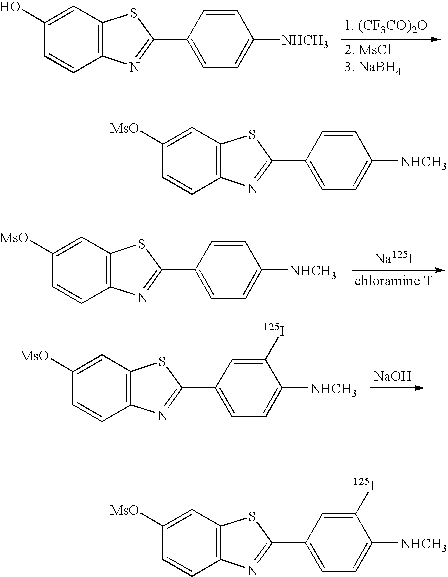

To a solution of 2-(4′-methylaminophenyl)-6-hydroxy benzothiazole (300 mg, 1.17 mmol) dissolved in CH₂Cl₂ (20 mL) was added Et₃N (2 mL) and trifluoroacetic acid (1.5 mL). The reaction mixture was stirred at room temperature for 3 h. The solvent was removed under reduced pressure and the residue was dissolved in ethyl acetate (30 mL), washed with NaHCO₃ solution. Brine, water, and dried over MgSO₄. After evaporation of the solvent, the residue was dissolved in acetone (20 ml, pre-dried over K₂CO₃), K₂CO₃ (1.0 g, powered) was added followed by MsCl (400 mg, 3.49 mmol). The reaction mixture was stirred at room temperature and monitored with TLC until starting material disappeared. The residue was then filtrated. The filtrate was evaporated under reduced pressure. The residue was dissolved in ethyl acetate (30 mL), washed with NaHCO₃ solution. Brine, water, and dried over MgSO₄. After evaporation of the solvent, the residue was dissolved in EtOH and NaBH₄ was added. The reaction mixture was stirred at room temperature for 2 h. The solvent was evaporated and the residue was dissolved in water, extracted with ethyl acetate (20 ml×3), the extracts were combined and dried over MgSO₄. After evaporation of the solvent, the residue was purified with flash column (hexanes/ethyl acetate=8:1) to give the product (184 mg, 47.0%) as brown solid. ¹HNMR (300 MHz, CDCl₃) δ (ppm): 7.94 (d, J=8.8 Hz, 1H), 7.87 (d, J=8.7 Hz, 2H), 7.77 (d, J=2.3 Hz, 1H), 7.30 (dd, J₁=8.8 Hz, J₂=2.3 Hz, 1H), 6.63 (d, J=8.7 Hz, 2H), 3.16 (s, CH₃), 2.89 (s, NCH₃).

### General Procedures for Radiolabelling:

To a solution of 2-(4′-aminophenyl)-6-methanesulfonoxy benzothiazole or 2-(4′-methylaminophenyl)-6-methylsulfoxy benzothiazole (1 mg) in 250 μL acetic acid in a sealed vial was added 40 μL of chloramines T solution (28 mg dissolved in 500 μL acetic acid) followed by 27 μL (ca. 5 mCi) of sodium [¹²⁵I]iodide (specific activity 2,175 Ci/mmol). The reaction mixture was stirred at r.t. for 2.5 hrs and quenched with saturated sodium hydrogensulfite solution. After dilution with 20 ml of water, the reaction mixture was loaded onto C8 Plus SepPak and eluted with 2 ml methanol. For deprotection of the methanesulfonyl group, 0.5 ml of 1 M NaOH was added to the eluted solution of radioiodinated intermediate. The mixture was heated at 50° C. for 2 hours. After being quenched by 500 μL of 1 M acetic acid, the reaction mixture was diluted with 40 mL of water and loaded onto a C8 Plus SepPak. The radioiodinated product, having a radioactivity of ca. 3 mCi, was eluted off the SepPak with 2 mL of methanol. The solution was condensed by a nitrogen stream to 300 μL and the crude product was purified by HPLC on a Phenomenex ODS column (MeCN/TEA buffer, 35:65, pH 7.5, flow rate 0.5 mL/min up to 4 min, 1.0 mL/min at 4-6 min, and 2.0 mL/min after 6 min, retention time 23.6). The collected fractions were loaded onto a C8 Plus SepPak. Elution with 1 mL of ethanol gave ca. 1 mCi of the final radioiodinated product.

### BIOLOGICAL EXAMPLE

### [¹¹C]PIB PET Study Compilation

### I. Arterial Based Methods

A. Study Participant Information

A total of 21 [¹¹C]PIB PET studies have been performed on 16 subjects. Five of the 21 studies were test/re-test studies. Table 1 lists subject characteristics including age, mini-mental state examination (MMSE) score and gender. Three of these subjects are from a large famial AD (FAD) kindred (highlighted in grey in Table 1; M+ indicates mutation carrier, S+ indicates symptomatic dementia). Subjects were recruited and evaluated, receiving their diagnosis in a consensus conference of experienced neurologists, psychiatrists, neuropsychologists and clinicians according to published criteria. (Lopez et al., Neurology 55:1854-1862, 2000).

Of the five control subjects, C-4 was a young control age-matched to the M+S−FAD subject and C-5 was a M−S-sibling of the AD-5 M+S+FAD patient. Of the 5 MCI subjects, MCI-2 and MCI-5 have been cognitively stable while the others have had slow, mild but progressive cognitive decline limited only to memory at the time of the [¹¹C]PIB study.

All subjects underwent fully-dynamic [¹¹C]PIB (90 min) and simplified FDG (25 min) PET imaging studies. The procedure was well-tolerated and completed by all subjects. All five subjects who were asked to return for a re-test within 21 days agreed and again completed the study without problems. The [¹¹C]PIB studies were conducted over 90 min after slow (20 sec) bolus injection of [¹¹C]PIB. PET scanning was performed using a Siemens/CTI HR+ scanner (see section C.2.3.2).

TABLE 1
Subject Characteristics

| | | | | |
|---|---|---|---|---|
| Status | Study | Age (yr) | MMSE | Gender |

Controls

| | | | | |
|---|---|---|---|---|
| C-1 | Test (1) | 65 | 30 | F |
| C-2 | Test & Retest | 76 | 30 | M |
| C-3 | Test & Retest | 69 | 30 | F |
| C-4 | Test (1) | 39 | 28 | M |
| C-5 (M − S−) | Test (1) | 45 | 29 | F |
| Mean | | 59 ± 16 | 29 ± 1 | |

Patients

| | | | | |
|---|---|---|---|---|
| AD-1 | Test & Retest | 58 | 18 | M |
| AD-2 | Test & Retest | 75 | 26 | M |
| AD-3 | Test (1) | 77 | 26 | M |
| AD-4 | Test (1) | 57 | 25 | M |
| AD-5 (M + S+) | Test (1) | 52 | 21 | M |
| Mean | | 64 ± 11 | 23 ± 4 | |
| MCI-1 | Test & Retest | 67 | 24 | M |
| MCI-2 | Test (1) | 77 | 29 | M |
| MCI-3 | Test (1) | 82 | 23 | M |
| MCI-4 | Test (1) | 74 | 27 | M |
| MCI-5 | Test (1) | 55 | 28 | M |
| Mean | | 71 ± 10 | 26 ± 3 | |

Other

| | | | | |
|---|---|---|---|---|
| M + S− (1) | Test (1) | 36 | 30 | F |

B. Blood Collection Data for Determining [¹¹C]PIB Radioactivity Concentration in Plasma

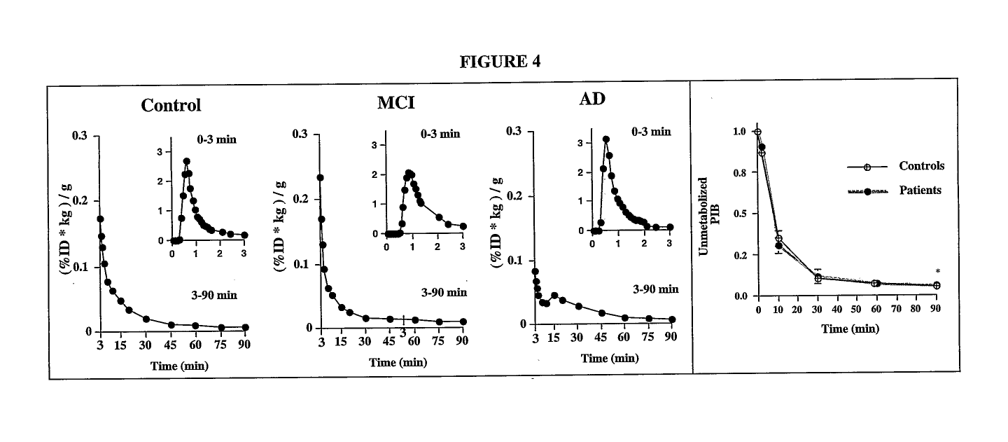

Arterial blood sampling was successfully performed in all 21 studies. The average [¹¹C]PIB radioactivity concentration in plasma is shown, in FIG. 4, for a representative control, MCI, and AD subject. The concentration of [¹¹C]PIB and the fraction of unmetabolized [¹¹C]PIB in plasma were similar between subjects (FIG. 4). The level of free [¹¹C]PIB in plasma (f1) was similar in controls (f1=0.132±0.009) and patients (f1=0.134:0.004). These f1 values are intermediate with respect to neuroreceptor-binding radiotracers that typically have values ranging from 0.06 ([¹¹C]WAY-100635, (Parsey et al., J. Cereb. Blood Flow Metab. 20:1111-1133, 2000), to 0.30 (diprenorphine), (Sadzot et al, J. Cereb. Blood Flow Metab. 11:204-219, 1991).

C. ROI-Based SUV Analyses

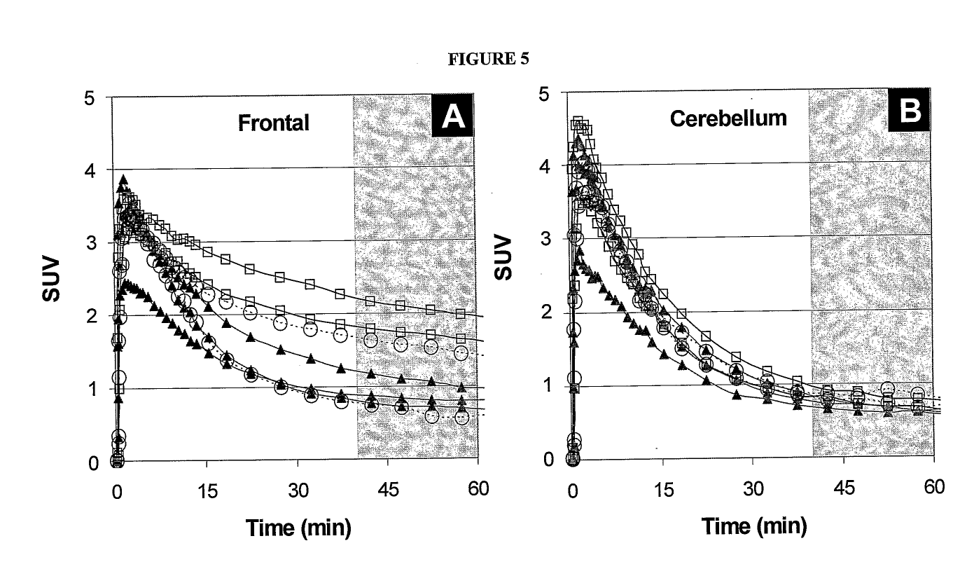

Examples of [¹¹C]PIB time-activity curves in terms of SUV data are shown in FIG. 5 for the frontal and cerebellar regions. For simplicity, only data from the first three controls and first two AD and MCI subjects are shown (parametric data from all subjects is shown in FIG. 3). Similar to initial studies performed, the present SUV data showed approximately two-fold higher [¹¹C]PIB retention in the two AD subjects compared to controls from 40-60 min. The MCI-1 subject (deteriorating) was intermediate, while the MCI-2 subject (cognitively stable) was indistinguishable from two of the control subjects (C-1 and C-3). The oldest control (C-2, 76 y/o) had a 40-60 min average SUV of 1.1, suggesting the possibility of significant amyloid deposition. The cerebellar data were similar across subjects.

D. Analysis of Amyloid Imaging Data for Distinguishing Control, AD and MCI Patients

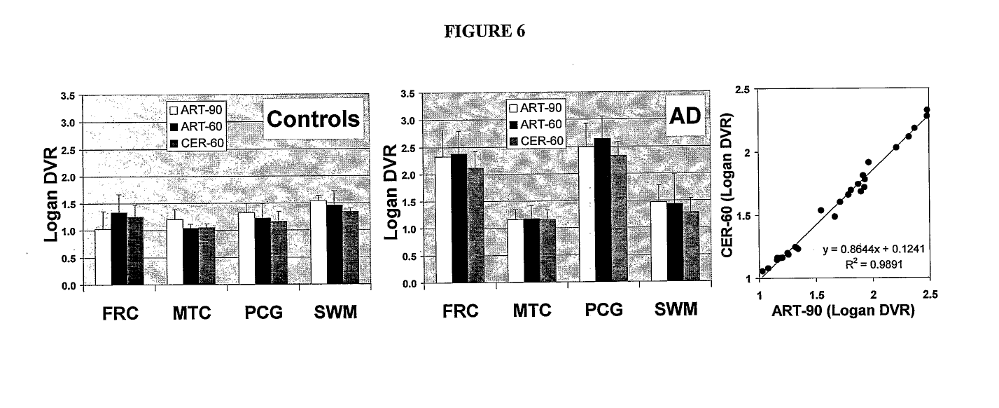

The preliminary [¹¹C]PIB data from the 5 controls, 5 AD and 5 MCI subjects and the one M+S−FAD subject studied to-date were analyzed using several methods over 60 and 90 min of data (all subjects scanned for 90 min). The analyses included 5- and 4-parameter, 3-compartmental models (including vascular volume) and the Logan graphical method with arterial blood data as the input function (Logan-ART) and with cerebellar data as the input function (Logan-CER). Also, a Patlak analysis and a 2-compartment model were applied but neither described the data as well as the 3-compartment or Logan methods based upon goodness-of-fit criteria and regression coefficients. Therefore, the Logan method was employed in the analyses discussed below. The Logan distribution volume ratios (DVRs) shown in FIG. 6 are the regional distribution values (DV) values normalized to the cerebellar DV (cerebellum as reference). The regional data were corrected for atrophy using ROI-based methods routinely employed. Meltzer et al., J Nucl. Med. 40:2053-2065 (1999).

Examples of the initial results obtained from the first 16 subjects using the Logan graphical method (using of arterial blood or cerebellar data as input functions) are shown in FIG. 6.

The data shown in FIG. 6 reveals:

1) The present pharmacokinetic analysis is consistent with the SUV analysis presented above. That is, the AD subjects clearly show higher DVR values than the control subjects in the cortical areas known to have heavy amyloid deposition, such as frontal and posterior cingulate cortex. Furthermore, AD patients and controls are equivalent in areas without neuritic plaques such as mesial temporal cortex and cerebellum; and

2) The Logan-CER-60 method produces results that are very similar to the 60 and 90 min arterial blood input methods. Although the DVR values determined with cerebellum as input and using only the first 60 min of data (Logan-CER-60) are systematically lower, the correlation with the 90 min arterial input data (Logan-ART-90) is very good (R²=0.989; FIG. 3).

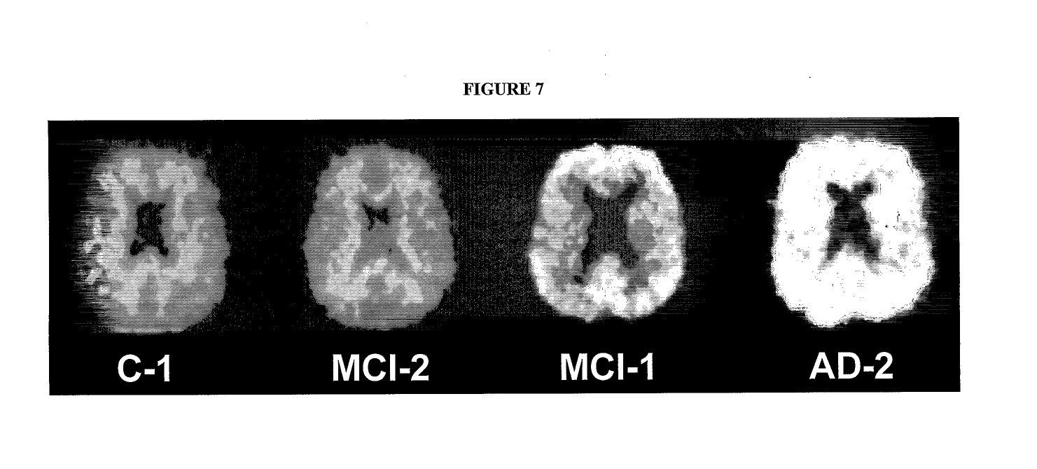

The Logan-CER-60 DVR data for all 5 controls, 5 MCI, 5 AD subjects and the one M+S−eFAD subject are shown in FIG. 3. The controls fall into a fairly narrow range from 1.0 to ˜1.5. This extends above the value of 1.0 which indicates [¹¹C]PIB kinetics equivalent to cerebellum. The younger controls fall consistently near DVR values of 1.0 and the oldest control, C-2, consistently falls near 1.5 in most cortical areas indicating a true determination of amyloid deposition in an asymptomatic subject. In all cortical areas except mesial temporal cortex (an area known to have little amyloid deposition. Arnold et al., Cereb. Cortex 1:103-116, (1991), the AD patients had DVR values typically between 1.5 and 2.5. The area with the highest [¹¹C]PIB Logan-CER-60 DVR was the posterior cingulate cortex that had a mean DVR two-fold that of the control DVR in this region (p=0.00007). AD and control were not distinguishable in the mesial temporal cortex, pons, subcortical white matter and cerebellum. This data indicates that any [¹¹C]PIB retention in these areas was non-specific. The M+S+FAD patient had relatively low [¹¹C]PIB retention in most cortical areas, but relatively higher [¹¹C]PIB retention in the caudate and occipital cortex. The M+S−subject from the same FAD family showed no evidence of [¹¹C]PIB retention. The MCI subjects had a mean DVR value that was intermediate between control and AD in most regions. Detailed inspection showed that the MCI subjects fell into two groups. The cognitively stable subjects (MCI-2 and MCI-5) are indistinguishable from control subjects in all brain areas. The other three MCI subjects are indistinguishable from the AD patients in all brain areas (FIG. 7). This data indicates that imaging with [¹¹C]PIB PET can accurately distinguish between MCI subjects with and without amyloid deposition.

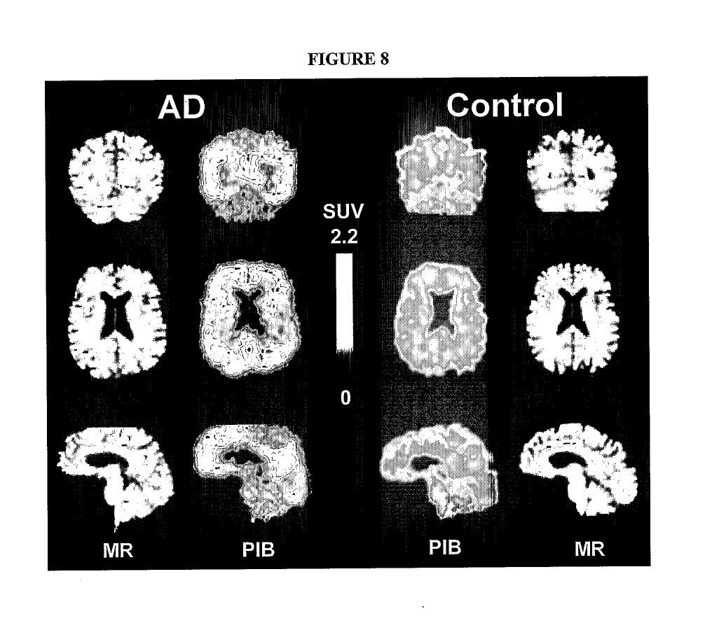

E. Voxel-Based Analyses

[¹¹C]PIB SUV images show marked [¹¹C]PIB retention in association cortices and little retention in cerebellum (FIG. 8). In addition, the [¹¹C]PIB and FDG image data were analyzed using arterial input function data with the Logan graphical and Hutchins FDG methods, respectively (lumped constant=1.0, see D2.3.5) Logan et al. J. Cereb. Blood Flow Metab. 16:834-840 (1996); Logan et al., J. Cereb. Blood Flow Metab. 10:740-747 (1990); Hutchins et al., J. Cereb. Blood Flow Metab. 4:35-40 (1984).

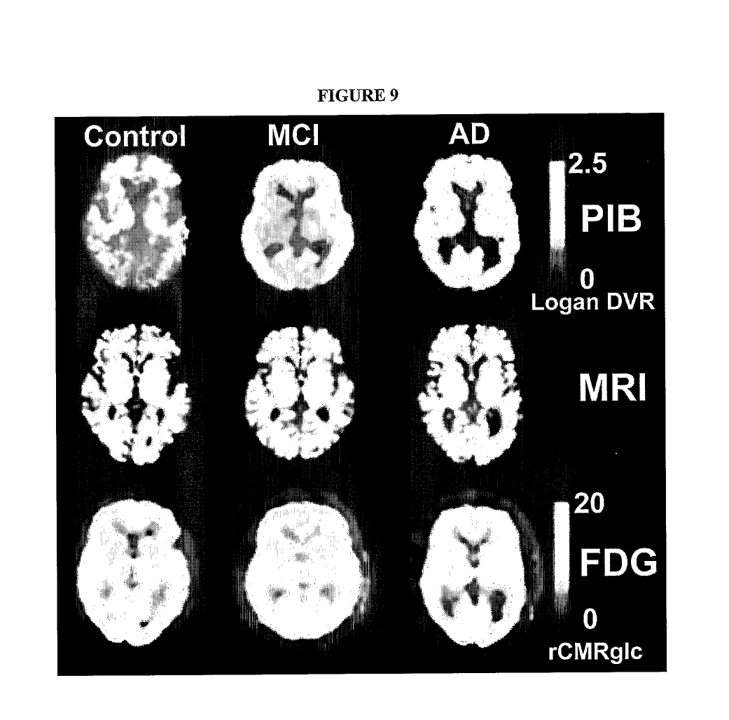

These image data were not corrected for cerebral atrophy but still demonstrate greater [¹¹C]PIB localization in cortical areas for the AD subject (FIG. 9) relative to MCI-1 (deteriorating) and the C-1 control. The FDG image data show the established area of hypo-metabolism in the parietal cortex of the AD subject (Small et al., 2000; Mielke et al., 1996). The MCI subject does not show abnormalities on the FDG scan.

F. Re-Test Reliability

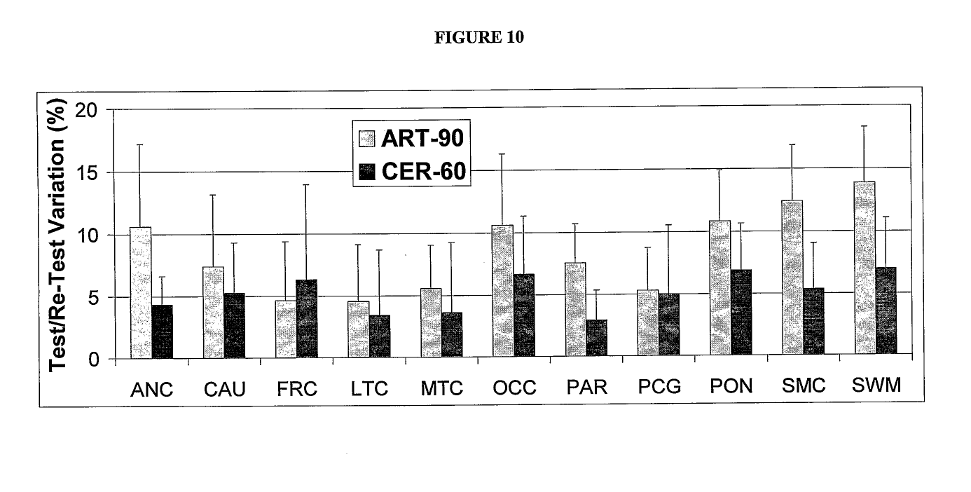

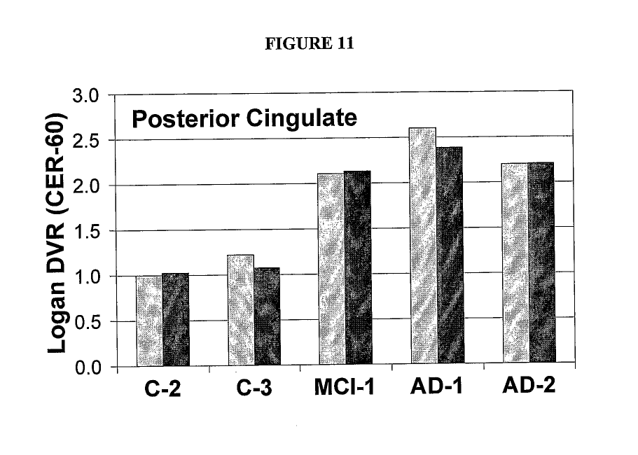

Both the Logan-ART-90 min method and the Logan-CER-60 min method proved to be very stable in a test/re-test study. The Logan-ART-90 method showed a mean test/re-test variation of 8.5±5.3% over all areas studied and the Logan-CER-60 method showed an even better test/re-test variation of 5.1±4.5% (FIG. 10). Test/re-test data for the posterior cingulate cortex of the five subjects is shown in FIG. 11. The stability is similar regardless of whether amyloid deposition (i.e., [¹¹C]PIB retention) is present.

### II. Simplified Non-Arterial-Based Methods of Analysis

This section describes efforts to extend the above-summarized, quantitative PIB studies, with nine additional subjects (n=24), to include an evaluation of simplified methodology, i.e., methods that do not require arterial blood sampling, for PIB PET imaging studies. In these examples, the performance of several methodological simplifications for PIB PET were compared to that of the fully-quantitative method-of-choice, Logan graphical analysis, based on arterial input and 90 minutes of emission data. (Logan-ART-90 min will be referred to as ART90 in this section.) The simplifications included a shorter scan duration, the use of image-derived cerebellar or carotid time-activity data, in lieu of an arterial input function, and a single-scan method based upon the ratio of standardized uptake values (SUV) in the region-of-interest normalized to the cerebellar SUV. These examples illustrate a PIB PET methodology that can be simply and reliably applied across the AD disease spectrum, while providing a good compromise between accuracy and precision in the PIB retention measures.

A. Human Subjects

PIB PET imaging was performed for 24 subjects, which included healthy controls (3M, 5F: 65±16 years), and subjects with a diagnosis of either MCI (8M, 2F: 72±9 yrs) or AD (6M: 67±10 yrs). Table 2 below describes the subject characteristics including age, MMSE score and gender. The procedure was well tolerated by all subjects.

TABLE 2
Subject Demographics

| | | | |
|---|---|---|---|
| Group | MMSE* | Gender | Study^{†} |

Controls

| | | | |
|---|---|---|---|
| C-1 | 30 | F | Test |
| C-2 | 30 | M | Test-Retest (18) |
| C-3 | 30 | F | Test-Retest (8) |
| C-4 | 28 | M | Test |
| C-5 | 29 | F | Test |
| C-6 | 29 | F | Test |
| C-7 | 30 | M | Test-Retest (21) |
| C-8 | 30 | F | Test-Retest (19) |

Mean Age: 65 ± 16 (72, 83, 70, 45, 39, 69, 76, 65)^{‡}

MCI Patients

| | | | |
|---|---|---|---|
| M-1 | 24 | M | Test-Retest (20) |
| M-2 | 29 | M | Test |
| M-3 | 23 | M | Test |
| M-4 | 27 | M | Test |
| M-5 | 28 | M | Test |
| M-6 | 29 | M | Test |
| M-7 | 29 | M | Test |
| M-8 | 27 | M | Test |
| M-9 | 29 | F | Test |
| M-10 | 29 | F | Test |

Mean Age: 72 ± 9 (74, 62, 80, 79, 65, 55, 74, 82, 77, 67)^{‡}

AD Patients

| | | | |
|---|---|---|---|
| A-1 | 18 | M | Test-Retest (18) |
| A-2 | 26 | M | Test-Retest (19) |
| A-3 | 26 | M | Test |
| A-4 | 25 | M | Test |
| A-5 | 26 | M | Test |
| A-6 | 25 | M | Test-Retest (28) |

Mean Age: 67 ± 10 (63, 73, 57, 77, 75, 58)^{‡}
*MMSE: Mini-mental State Examination (37)
^{†}Eight subjects underwent a second “re-test” PIB PET study within 28 days of the “test” or baseline study. The time interval between scans is shown in parentheses
^{‡}Individual ages are listed in parentheses. Ages are not listed in the order of the subject ID to preserve anonymity.

B. Imaging

High specific activity (SA) PIB PET studies were performed in the 8 healthy controls (dose: 488.4±107.3 MBq; SA: 47.8±21.7 GBq/μmol), 10 MCI patients (dose: 510.6±77.7 MBq; SA: 45.9±24.9 GBq/μmol), and 6 AD patients (dose: 514.3±96.2 MBq; SA: 31.3±18.1 GBq/μmol). Average regional CSF factors are shown in Table 3 below. The regional CSF correction factors, which were determined from each individual subject's SPGR MR data, showed no significant differences for any group comparison, using the one-sided non-parametric Wilcoxon rank test after FDR correction.

TABLE 3
Average Regional CSF Correction factors

| | | ACG | CAU | CER | FRC | LTC | MTC | OCC | PAR | PCG | PON | SMC | SWM |
|---|---|---|---|---|---|---|---|---|---|---|---|---|---|
| Controls | mean | 0.91 | 0.86 | 0.96 | 0.86 | 0.91 | 0.94 | 0.91 | 0.85 | 0.90 | 0.98 | 0.79 | 0.99 |
| | s.d. | 0.05 | 0.05 | 0.03 | 0.06 | 0.04 | 0.03 | 0.06 | 0.10 | 0.09 | 0.01 | 0.09 | 0.00 |
| MCI | mean | 0.80 | 0.84 | 0.95 | 0.82 | 0.85 | 0.84 | 0.87 | 0.81 | 0.88 | 0.97 | 0.74 | 0.99 |
| | s.d. | 0.09 | 0.03 | 0.03 | 0.04 | 0.07 | 0.08 | 0.05 | 0.10 | 0.06 | 0.01 | 0.06 | 0.01 |
| AD | mean | 0.82 | 0.84 | 0.96 | 0.83 | 0.85 | 0.89 | 0.91 | 0.80 | 0.86 | 0.98 | 0.74 | 0.99 |
| | s.d. | 0.08 | 0.05 | 0.03 | 0.06 | 0.07 | 0.05 | 0.05 | 0.12 | 0.09 | 0.01 | 0.07 | 0.01 |

C. Input Function Comparisons

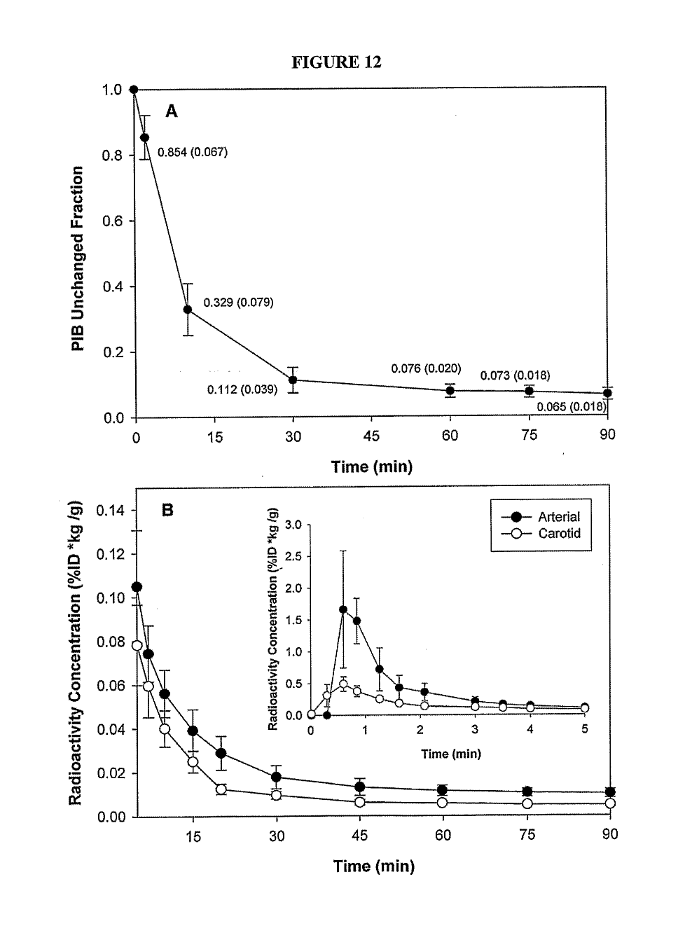

The input functions determined via hand-drawn arterial samples were compared to those derived by carotid VOI placement. Metabolite-corrected input functions determined by arterial sampling and carotid VOI placement were corrected for injected dose and body mass (% ID*kg/g) to allow population-average input functions (n=24) to be generated for the purpose of comparison (FIG. 12). Arterial input functions were interpolated to the frame midpoint times of the PET emission images for the Logan graphical analysis, which permitted direct comparison with the carotid-based input function. The average arterial input function was found to peak at a value of 1.66±0.92% ID*kg/g, while the average carotid input function peaked at a value of 0.49±0.11% ID*kg/g. The peak value in both cases occurred in the third frame of acquisition (midpoint: 36 sec post-injection). At early times (<5 min) the carotid input function underestimated the arterial input function by as much as a factor of 4 on average. At later times (>5 min), the carotid input function reflected the shape of the arterial input function more closely, converging somewhat with the latter to maintain a constant ratio between the methods of 2.

D. Data Analysis

This section includes a description of the basic PET data, a summary of the primary results that were observed across all methods, method-specific performance issues, and evaluations of method performance. Comparisons of the mean PIB retention measures focused on differences between AD and control subject groups because PIB retention for the ten MCI subjects was found to range across control and AD levels. That is, MCI subjects do not represent a homogeneous group distinct from either controls or AD subjects.

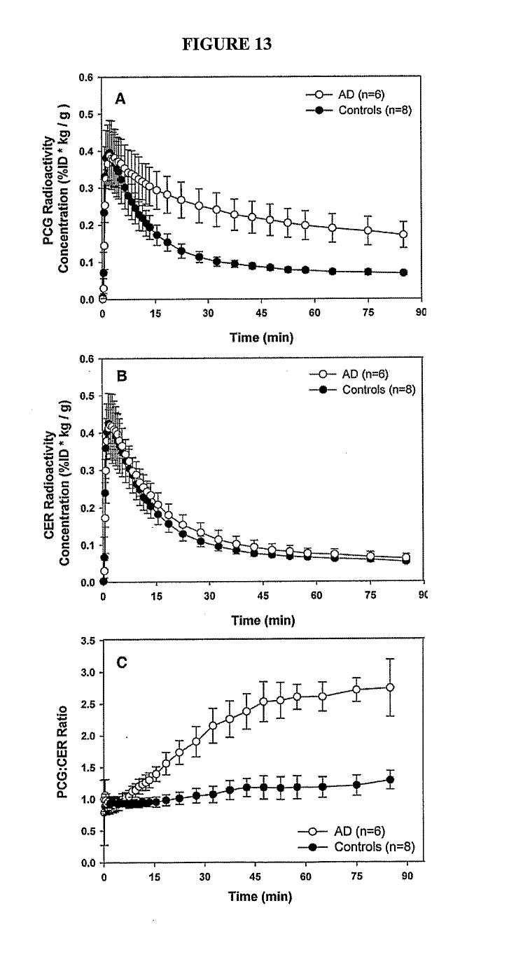

Tissue Data. Tissue:cerebellar radioactivity concentration ratios were computed for each brain region. In posterior cingulate, the region that showed the highest degree of PIB retention in AD subjects, the VOI:CER ratios reached a plateau at a value of approximately 2.5:1 after 45 min while control subjects maintained ratios of approximately 1:1 for all primary amyloid-binding areas (FIG. 13). On average, the tissue:CER ratios began to plateau at about 20 min in controls and 45-50 min in AD subjects.

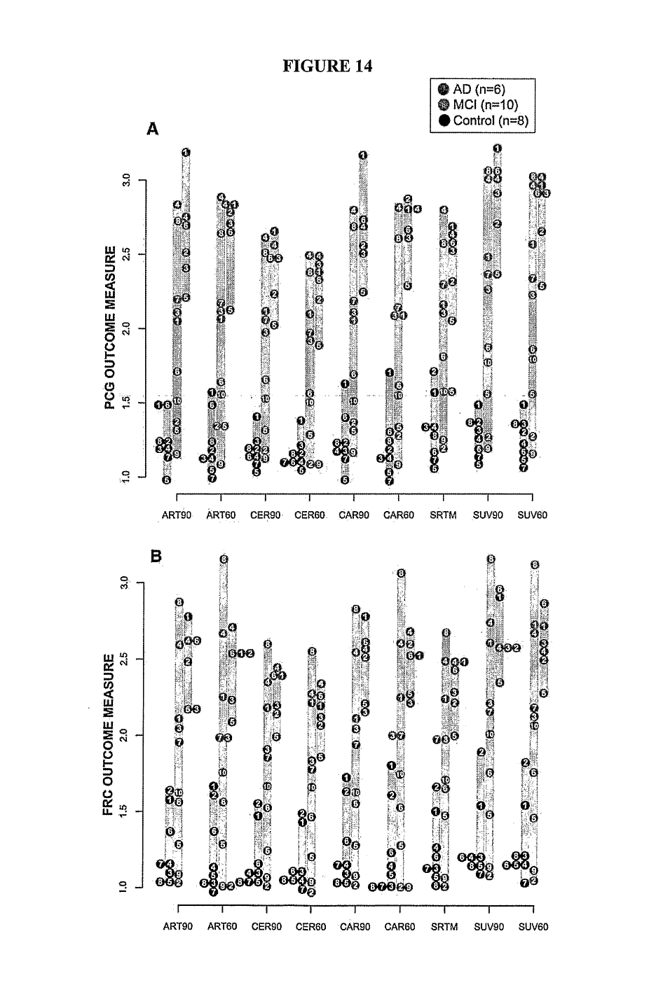

Overall Results. Table 4 below lists the mean values measured in AD and control subjects, for each method, across the 11 regions. All methods yielded significantly higher DVR or SUVR values for AD subjects compared to controls in regions known to contain amyloid in AD. The most significant differences (p<0.001, see statistical methods) were generally observed in PCG, ACG, FRC, PAR, LTC, CAU (Table 4). Lesser differences (0.001≦p≦0.05) were observed for OCC, SMC, and MTC. There were no significant differences in PIB retention between AD and control subjects in regions that are known to be virtually free of amyloid pathology in mild-to-moderate AD subjects, such as SWM and PON (p>0.20). No method yielded significant group differences in the cerebellar DV or SUV value for AD patients relative to controls (p>0.25). FIG. 14 shows scatter plots of the individual subject DVR and SUVR values, for the posterior cingulate and frontal areas, for the various analysis methods and subject groups.

TABLE 4
Outcome Measures*

| | | | | | | | | | |
|---|---|---|---|---|---|---|---|---|---|
| | ART90 | ART60 | CER90 | CER60 | CAR90 | CAR60 | SRTM90 | SUVR90 | SUVR60 |
| REGION | DVR | DVR | DVR | DVR | DVR | DVR | BP + 1 | Ratio | Ratio |

Controls

| | | | | | | | | | |
|---|---|---|---|---|---|---|---|---|---|
| PCG | 1.24 (13.6) | 1.22 (16.9) | 1.18 (9.1) | 1.15 (8.9) | 1.25 (15.6) | 1.21 (18.2) | 1.32 (17.1) | 1.27 (10.4) | 1.25 (11.2) |
| ACG | 1.26 (18.4) | 1.23 (22.6) | 1.19 (14.8) | 1.15 (13.9) | 1.26 (21.0) | 1.24 (24.9) | 1.24 (18.2) | 1.32 (19.8) | 1.3 (18.7) |
| PAR | 1.31 (11.5) | 1.30 (13.2) | 1.24 (9.7) | 1.21 (9.6) | 1.31 (14.5) | 1.30 (16.3) | 1.32 (13.7) | 1.35 (9.7) | 1.33 (9.2) |
| FRC | 1.26 (18.9) | 1.24 (22.2) | 1.19 (16.6) | 1.16 (16.0) | 1.26 (21.3) | 1.24 (24.5) | 1.25 (17.6) | 1.30 (21.0) | 1.28 (20.5) |
| LTC | 1.21 (10.0) | 1.20 (11.7) | 1.17 (7.0) | 1.14 (6.6) | 1.22 (11.5) | 1.20 (13.4) | 1.16 (9.5) | 1.27 (9.4) | 1.26 (8.8) |
| CAU | 1.05 (9.2) | 0.98 (12.4) | 1.07 (7.3) | 1.04 (6.1) | 1.07 (11.0) | 1.01 (15.6) | 1.13 (12.5) | 1.14 (11.7) | 1.11 (11.4) |
| SMC | 1.25 (9.9) | 1.25 (13.6) | 1.23 (8.6) | 1.21 (7.6) | 1.27 (12.8) | 1.27 (14.9) | 1.35 (22.8) | 1.32 (9.8) | 1.30 (9.3) |
| OCC | 1.28 (12.3) | 1.25 (14.8) | 1.17 (6.6) | 1.14 (6.1) | 1.28 (13.1) | 1.24 (14.1) | 1.2 (11.2) | 1.25 (7.5) | 1.22 (7.5) |
| MTC | 0.99 (5.9) | 0.98 (7.5) | 1.02 (6.2) | 1.00 (5.9) | 1.01 (8.8) | 0.99 (9.6) | 1.04 (11.7) | 1.11 (7.8) | 1.12 (8.1) |
| SWM | 1.42 (12.0) | 1.36 (16.7) | 1.33 (4.6) | 1.25 (7.9) | 1.43 (11.0) | 1.35 (20.4) | 1.35 (7.3) | 1.69 (7.1) | 1.66 (7.4) |
| PON | 1.40 (8.1) | 1.42 (8.8) | 1.47 (5.6) | 1.47 (6.1) | 1.42 (6.6) | 1.46 (7.6) | 1.41 (12.9) | 1.76 (8.2) | 1.80 (7.3) |
| CER DV | 3.75 (15.2) | 3.55 (19.7) | | | 7.26 (9.0) | 6.72 (11.0) | | | |

AD

| | | | | | | | | | |
|---|---|---|---|---|---|---|---|---|---|
| PCG | 2.63 (12.9)^{‡} | 2.66 (10.2)^{‡} | 2.41 (10.0)^{‡} | 2.29 (9.6)^{‡} | 2.65 (11.5)^{‡} | 2.67 (7.8)^{‡} | 2.47 (9.6)^{‡} | 2.88 (10.4)^{‡} | 2.80 (9.9)^{‡} |
| ACG | 2.52 (15.9)^{‡} | 2.57 (19.2)^{‡} | 2.33 (14.2)^{‡} | 2.23 (15.5)^{‡} | 2.53 (15.2)^{‡} | 2.58 (17.0)^{‡} | 2.45 (14.3)^{‡} | 2.82 (15.4)^{‡} | 2.75 (14.9)^{‡} |
| PAR | 2.50 (16.8)^{‡} | 2.48 (18.1)^{‡} | 2.29 (16.2)^{‡} | 2.17 (17.1)^{‡} | 2.50 (15.8)^{‡} | 2.50 (15.8)^{‡} | 2.33 (16.2)^{‡} | 2.72 (15.9)^{‡} | 2.65 (15.6)^{‡} |
| FRC | 2.47 (10.5)^{‡} | 2.44 (9.5)^{‡} | 2.26 (8.0)^{‡} | 2.14 (7.9)^{‡} | 2.48 (9.8)^{‡} | 2.47 (7.7)^{‡} | 2.32 (8.3)^{‡} | 2.66 (8.8)^{‡} | 2.58 (7.7)^{‡} |
| LTC | 2.35 (16.6)^{‡} | 2.34 (14.9)^{‡} | 2.16 (13.0)^{‡} | 2.06 (12.5)^{‡} | 2.35 (16.1)^{‡} | 2.36 (12.6)^{‡} | 2.16 (13.1)^{‡} | 2.55 (14.6)^{‡} | 2.49 (12.8)^{‡} |
| CAU | 2.02 (18.3)^{‡} | 2.00 (19.0)^{‡} | 1.96 (15.3)^{‡} | 1.89 (15.8)^{‡} | 2.02 (17.5)^{‡} | 2.01 (18.1)^{‡} | 2.08 (19.0)^{‡} | 2.34 (17.6)^{‡} | 2.31 (16.5)^{‡} |
| SMC | 1.84 (20.7)^{‡} | 1.77 (23.0)^{†} | 1.73 (18.5)^{†} | 1.65 (19.0)^{†} | 1.83 (20.0)^{†} | 1.78 (22.2)^{†} | 1.93 (18.9)^{†} | 1.99 (21.8)^{†} | 1.93 (20.9)^{†} |
| OCC | 1.75 (17.7)^{‡} | 1.66 (14.9)^{†} | 1.62 (14.8)^{‡} | 1.53 (13.5)^{‡} | 1.75 (17.6)^{‡} | 1.66 (14.3)^{†} | 1.81 (17.4)^{‡} | 1.83 (18.6)^{‡} | 1.78 (16.4)^{‡} |
| MTC | 1.23 (13.8)^{‡} | 1.20 (16.8)^{†} | 1.20 (10.8)^{†} | 1.16 (12.4)^{†} | 1.23 (13.4)^{†} | 1.21 (16.1)^{†} | 1.22 (10.8)^{†} | 1.40 (14.5)^{†} | 1.38 (13.5)^{†} |
| SWM | 1.52 (13.2) | 1.44 (36.0) | 1.34 (13.4) | 1.26 (18.5) | 1.51 (14.0) | 1.39 (44.1) | 1.36 (10.0) | 1.61 (11.5) | 1.56 (13.0) |
| PON | 1.40 (7.7) | 1.47 (4.2) | 1.44 (6.4) | 1.44 (6.0) | 1.40 (7.7) | 1.47 (3.3) | 1.44 (6.8) | 1.69 (7.2) | 1.73 (6.7) |
| CER DV | 3.70 (15.4) | 3.38 (13.9) | — | — | 8.53 (12.6) | 7.86 (10.7) | — | — | — |

*Values presented represent mean (coefficient of variation (%))
^{†}p < 0.05, AD < controls, 1-sided
^{‡}p < 0.001, AD < controls, 1-sided

Three of the MCI subjects (M-2, 5, 9) showed patterns of PIB retention that were indistinguishable from the control group. Five MCI subjects (M-1, 3, 4, 7, 8) demonstrated patterns of retention that were characteristic of the AD subject group. Two MCI subjects (M-6, 10) tended to be higher than controls in PCG or FRC (FIG. 14).

Standardized uptake value (SUV). The single (summed) scan tissue ratios that were computed over either 40-60 min (SUVR60) or 40-90 min (SUVR90) were found to be in agreement for both the AD and control subject groups. In controls, the regional SUVR60 ratios ranged from 1.11±0.13 (CAU) to 1.80±0.13 (PON), while the SUVR90 tissue ratios ranged from 1.14±0.13 (CAU) to 1.76±0.14 (PON). In AD subjects, the regional SUVR60 and SUVR90 values ranged from 1.38±0.19 (MTC) to 2.80±0.28 (PCG) and from 1.40±0.20 (MTC) to 2.88±0.30 (PCG), respectively.

Logan Graphical Analyses. The Logan graphical analysis generally provided estimates of DV (arterial or carotid input) and DVR (cerebellar input) values with high regression correlations (r²>0.97) in 10 of 11 regions. These results are consistent with the data satisfying the linearity condition required by the Logan analysis. For the SWM, correlations were generally lower (0.7<r²<0.99) than for other regions, particularly when the dataset was truncated to 60 min.

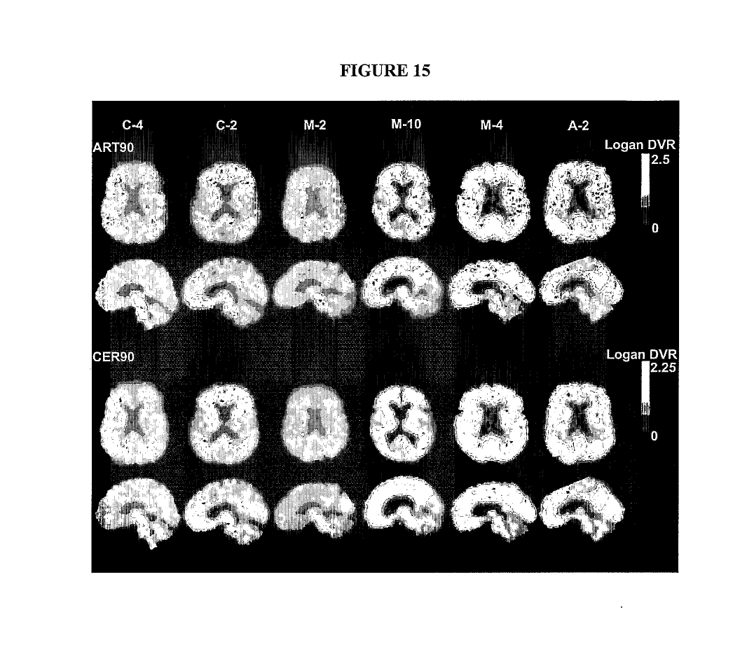

Parametric images of DVR measures obtained using the ART90 and CER90 analyses show similar patterns and levels of PIB retention (FIG. 15) in a normal control (C-4), a control with evidence of FRC amyloid deposition (C-2), an MC1 subject with no significant amyloid deposition (M-2), an MC1-subject with intermediate levels of PIB retention (M-10), an MC1-subject with a characteristic AD pattern of PIB retention (M-4), and a representative AD subject (A-2).

Multilinear Regression. The multilinear regression analysis (MA1) was applied using a reference tissue input in an exploratory manner for a high binding and low binding region (PCG and MTC) over 90 min. The MAI DVR estimates in these regions were essentially identical to those determined using CER90. This suggests that noise-induced bias is not a factor at the VOI level for the determination of the PIB Logan DVR. As a result of this excellent agreement, the remainder of the examples focus solely on Logan analysis results.

Simplified Reference Tissue Analysis. The use of SRTM with only 60 min of data resulted in highly variable outcome measures, spuriously overestimated values, and deviations in regional rank order. For this reason, typical SRTM results were obtained using 90 min of data. SRTM90 detected significant differences (p<0.001) in DVR values between control and AD subjects in several cortical and subcortical regions (Table 4). For the 90 min data set, average R_{I} values in control subjects ranged from 0.40±0.20 (SWM) to 0.99±0.15 (OCC). R_{I} values in AD subjects were comparable to controls in most regions, ranging from 0.35±0.08 (SWM) to 0.97±0.09 (OCC). In both AD and control subjects, only MTC and SWM showed R_{I} values consistently lower than 0.75. The most notable group difference in average R_{I} values was evident for PAR (controls: 0.86±0.06 and AD: 0.74±0.08), while PCG was more similar (controls: 0.91±0.06 and AD: 0.85±0.10). The aforementioned R_{I} values were not corrected for partial volume effects.

E. Evaluation Criteria

Rank Order. The regional rank order of outcome measures averaged for the six AD subjects was well conserved across all nine simplified methods as each identified PCG as the region with the greatest PIB retention, followed by ACG and other cortical regions, including PAR, FRC, and LTC (Table 5).

TABLE 5
Average Rank Order of Regional Outcome Measures by Method of Analysis*

| | METHOD | ACG | CAU | FRC | LTC | MTC | OCC | PAR | PCG | PON | SMC | SWM |
|---|---|---|---|---|---|---|---|---|---|---|---|---|
| Controls | ART90 | 6 | 10 | 7 | 9 | 11 | 4 | 3 | 8 | 1T | 5 | 1T |
| | ART60 | 9 | 11 | 7T | 7T | 10 | 4 | 3 | 6 | 1 | 5 | 2 |
| | CER90 | 5T | 10 | 9 | 5T | 11 | 5T | 3 | 8 | 1 | 4 | 2 |
| | CER60 | 8 | 10 | 9 | 6T | 11 | 6T | 2 | 5 | 1 | 4 | 3 |
| | CAR90 | 6 | 10 | 7T | 9 | 11 | 4 | 3 | 7T | 1 | 5 | 2 |
| | CAR60 | 8 | 10T | 7 | 9 | 10T | 5 | 2T | 6 | 1 | 4 | 2T |
| | SRTM90 | 6T | 10 | 8 | 9 | 11 | 6T | 3 | 5 | 1 | 4 | 2 |
| | SUVR90 | 5 | 10T | 8 | 6T | 10T | 9 | 3 | 6T | 1 | 4 | 2 |
| | SUVR60 | 5 | 11 | 8 | 6 | 10 | 9 | 3 | 7 | 1 | 4 | 2 |
| AD | ART90 | 2 | 6 | 3 | 5 | 11 | 8 | 4 | 1 | 10 | 7 | 9 |
| | ART60 | 2 | 6 | 4 | 5 | 11 | 8 | 3 | 1 | 10 | 7 | 9 |
| | CER90 | 2 | 6 | 3 | 5 | 11 | 7T | 4 | 1 | 9 | 7T | 10 |
| | CER60 | 2 | 6 | 3 | 5 | 11 | 8 | 4 | 1 | 9 | 7 | 10 |
| | CAR90 | 2T | 6 | 2T | 5 | 11 | 8 | 4 | 1 | 10 | 7 | 9 |
| | CAR60 | 2T | 6 | 4 | 5 | 11 | 8 | 2T | 1 | 10 | 7 | 9 |
| | SRTM90 | 2 | 6 | 3T | 5 | 11 | 8 | 3T | 1 | 9 | 7 | 10 |
| | SUVR90 | 2 | 6 | 3 | 5 | 11 | 8 | 4 | 1 | 9 | 7 | 10 |
| | SUVR60 | 2 | 6 | 3T | 5 | 11 | 9 | 3T | 1 | 8 | 7 | 10 |

*“T” designates a tie in the average ranking

PIB binding in caudate exceeded that of SMC, OCC, and MTC. White-matter-containing regions, such as PON and SWM, were among the lowest in terms of regional rank order in AD subjects. In control subjects, white-matter containing regions such as PON and SWM occupied the highest ranks.

The individual subject rank order was well-maintained across methods and regions. In general, CAR90 showed the best agreement with ART90 in terms of individual subject rank order (FIGS. 14A and 14B), although all simplified methods completely separated AD and control subjects by their respective outcome measures (DVR or SUVR) and no method resulted in subject misclassification. Also, all simplified methods distinguished the “AD-like” MCI subjects (M-1, 3, 4, 7, and 8) from the “control-like” MCI subjects (M-2, 5, and 9) consistently in both FRC and PCG (FIGS. 14A and 14B). However, discrepancies in the subject rank order were observed between ART90 and some simplified methods. For instance, ART90 and CAR90 identified subject A-1 as the AD subject with the greatest degree of PIB retention in PCG, which was far in excess of that observed for all other AD subjects (FIG. 14A). The CER90, SRTM, and SUVR90 methods also showed A-1 as having the highest degree of PIB retention in PCG, though by a smaller margin.

Methods that involved the truncation of the dataset to 60 min (ART60, CER60, CAR60, SUVR60) identified other subjects, A-4 or A-2, as the AD subject with the greatest PIB retention rather than A-1. Among the control subjects, the ART90 DVR values indicate subjects C-1 and C-6 to have elevated levels of PIB retention relative to other controls in PCG, while subjects C1-1 and C1-2 appear to have elevated PIB retention in FRC (FIGS. 14A and 14B). All simplified methods examined distinguished C-1 from other controls in both PCG and FRC, and C-2 in FRC. However, only ART60 agreed with ART90 with regard to the elevated status of C-6. Interestingly, inspection of the late summed PET images showed only subjects C-1 and C-2, among controls, to have a visually discernible pattern of cortical PIB retention suggestive of early AD, which even then was limited to FRC (FIG. 15).

Test-Retest Variability. The intra-subject, or test-retest, variability of the simplified PIB retention measures was evaluated for the eight subjects retested within 28 days of the initial PIB PET scan, using the percent difference and ICC measures (see Statistical methods). Table 6 summarizes the variability measures and shows that favorable margins of test-retest variability were observed that were generally within ±10% across methods and regions, except for SWM (6.0-23.8%).

TABLE 6
Test-Retest Variability of Simplified Methods of Analysis*^{,†}

| Method | ACG | CAU | FRC | LTC | MTC | OCC | PAR | PCG | PON | SMC | SWM | Avg | SD |
|---|---|---|---|---|---|---|---|---|---|---|---|---|---|
| ART90 | 7.5 | 8.6 | 4.9 | 3.6 | 7.1 | 7.7 | 4.6 | 5.2 | 7.8 | 7.2 | 11.5 | 6.9 | 2.2 |
| ART60 | 8.8 | 13.8 | 5.9 | 5.9 | 6.8 | 9.1 | 5.0 | 7.9 | 8.3 | 6.1 | 23.8 | 9.2 | 5.4 |
| CER90 | 4.0 | 5.6 | 5.6 | 2.9 | 3.9 | 4.8 | 3.0 | 2.7 | 5.6 | 4.9 | 7.8 | 4.6 | 1.5 |
| CER60 | 3.5 | 4.7 | 5.9 | 2.4 | 3.6 | 4.0 | 3.0 | 4.4 | 4.7 | 4.5 | 7.7 | 4.4 | 1.4 |
| CAR90 | 8.2 | 7.9 | 5.9 | 4.0 | 6.0 | 8.7 | 5.5 | 5.5 | 7.8 | 7.9 | 10.8 | 7.1 | 1.9 |
| CAR60 | 12.3 | 19.2 | 11.0 | 7.9 | 10.3 | 12.5 | 8.2 | 12.8 | 14.0 | 10.1 | 23.4 | 12.9 | 4.6 |
| SRTM90 | 4.4 | 6.3 | 5.9 | 4.0 | 6.5 | 6.9 | 4.2 | 5.8 | 9.9 | 7.7 | 7.0 | 6.2 | 1.7 |
| SUVR90 | 4.0 | 6.3 | 6.9 | 3.6 | 4.5 | 6.1 | 4.2 | 3.3 | 4.8 | 4.9 | 6.0 | 5.0 | 1.2 |
| SUVR60 | 4.3 | 5.0 | 8.0 | 4.1 | 4.6 | 5.4 | 4.3 | 4.9 | 4.5 | 5.1 | 7.7 | 5.3 | 1.3 |

*Test-retest variability computed as: ± |(Retest − Test)/Test *100|
^{†}Primary areas of interest are shown in boldface.

For most regions, the CER60 and CER90 methods showed lowest test-retest variability with averages within ±4.4% and ±4.6% respectively. Interestingly, the cerebellar-based SRTM method showed somewhat greater variation than either CER60 or CER90, averaging ±6.2% across all regions. The SUV-based methods were reproducible as well, averaging ±5.3% and ±5.0% across regions for SUVR60 and SUVR90, respectively. The greatest test-retest variability (within 10%) was observed for the arterial based methods. Greater variability was observed with a shorter scan duration, as is the case for CAR60 (+12.9%) and ART60 (9.2%), while that for the 90 min measures was less. ART90 and CAR90 performed similarly well, with test-retest variability across the 11 regions averaging ±6.9% and ±7.1%, respectively.

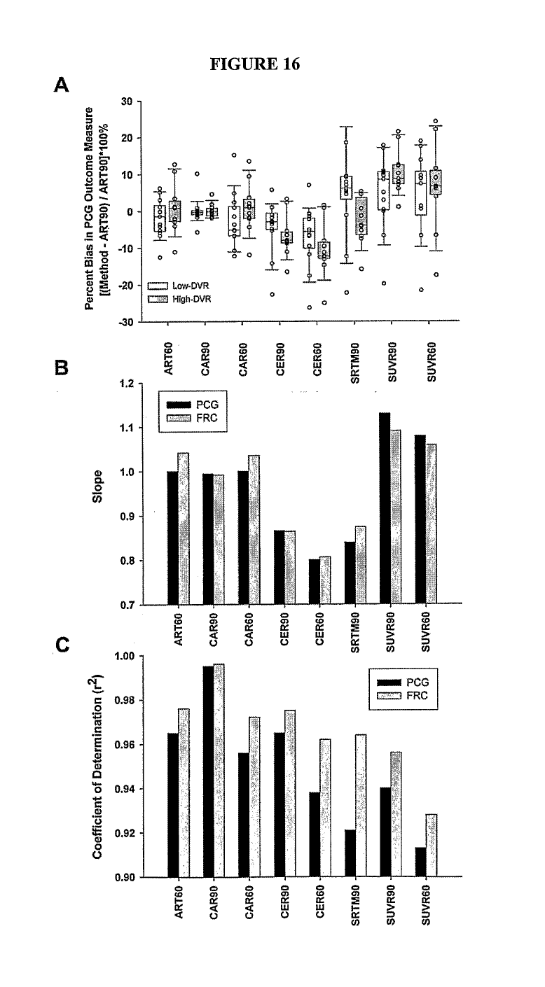

Bias and Correlation: Bias in the PIB retention measures was examined over low-DVR (ART90 PCG DVR<1.8, n=13) and high-DVR (ART90 PCG DVR>1.8, n=11) groups (see FIG. 14A). Box plots of the individual and mean % bias measures are shown for PCG in FIG. 16A. The lowest and most uniform % bias, across the low- and high-DVR data, was observed for the arterial-based methods. Greater % biases were observed for the SUVR and CER results. The CAR90PCG DVR measures most closely agreed with ART90 PCG DVR measures in low-DVR (% bias=0.11±3.44%) and high-DVR (% bias=0.19±1.86%) subjects. Slightly greater % bias and variation in this % bias was observed for the shorter scan duration methods of ART60 and CAR60, which was −1.79±5.20% and −2.57±7.23% for low-DVR subjects, respectively, and 0.75±6.66% and 1.00±6.72% for the high-DVR subjects, respectively. The CER methods showed the greatest negative % bias and greater negative % bias was observed for the high-DVR group relative to the low-DVR group. The SUVR methods showed the greatest positive % bias, but the % bias was fairly similar in the low- and high-DVR subjects. For a given method, the largest difference in % bias between low-DVR and high-DVR groups was found for SRTM90 (low: 6.03±14.47%; high: −2.65±6.37%). A similar pattern of results was observed for other cortical regions.

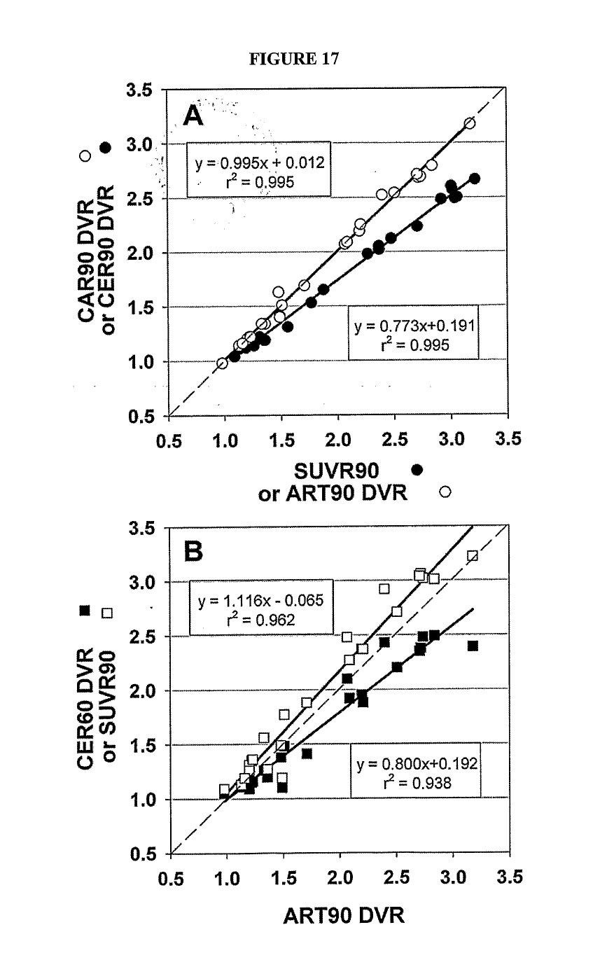

Across all subjects (n=24), the PCG and FRC DVR values determined using each simplified method were highly correlated (r²=0.921-0.995) with the ART90 DVR values (FIG. 16C). CAR90 produced near perfect correlations with ART90 (r²=0.995; slope=0.995; FIG. 17A). Of the methods examined, the SUVR60 results correlated most poorly with ART90 (r²=0.913; slope=1.083; FIGS. 16B & 16C), the CER60 method had the lowest slope (r²=0.938; slope=0.800; FIG. 17B) and the SUVR90 method had the highest slope (r²=0.962; slope=1.116; FIG. 17B). In an effort to determine if shared “noise” could lead to the good correlation between arterial methods and poorer correlation with other methods, we compared two non-arterial methods, CER90 and SUVR90, to each other. A correlation as strong as that between the arterial methods (r²=0.995, FIG. 17A) was found, although the slope of this correlation was relatively low (slope=0.773) suggesting a high bias between these methods. Overall, regression slopes ranged from 0.80-1.13 (FIG. 16B). The cerebellar-based methods tended to yield lower slopes (0.866-0.800; FIG. 16B), while the SUVR methods yielded higher values (1.073-1.116; FIG. 16B). The regression slopes tracked closely with % bias with the exception of the SRTM90 method (compare FIGS. 16A & 16B).

Effect Size: The effect size measure reflects the level of variation of a given measure across subjects (inter-subject variability) and separation of the group mean PIB retention values. It was often noted that arterial-based methods tended to be more variable than cerebellar-based methods and the 60 min data tended to be more variable than the 90 min data. For the controls, CER60 was generally associated with the least variation in DVR across subjects that was less than 10% for all regions except ACG (14%) and FRC (16%). ART60, CAR60, and SRTM90 yielded CV % values that were greater than 10% for 9 of 11 regions (excluding cerebellum) (Table 3). For the AD group, greater DVR coefficients of variation were most often observed for ART90 and ART60 ranging from about 10-20% in primary areas-of-interest.

All methods consistently separated control and AD groups and resulted in large Cohen's effect sizes for regions with high PIB retention. The greatest Cohen's effect sizes (d) were observed in the PCG and ranged from about 6.9 (SUV methods) to 4.6 (SRTM90). The magnitude of the effect sizes reflects that clear separation of mean PIB retention values is achieved between control and AD subjects. Table 7 lists the range of effect sizes in PCG, FRC, MTC, and PON.

TABLE 7
Effect Size Measurement for Selected Regions*

| Method | PON | MTC | FRC | PCG |
|---|---|---|---|---|
| ART90 | 0.0 | 1.9 | 4.5 | 5.1 |
| ART60 | 0.4 | 1.5 | 4.3 | 5.7 |
| CER90 | −0.3 | 1.8 | 5.2 | 6.5 |
| CER60 | −0.3 | 1.5 | 5.1 | 6.6 |
| CAR90 | −0.2 | 1.6 | 4.4 | 5.3 |
| CAR60 | 0.1 | 1.4 | 4.4 | 6.3 |
| SRTM90 | 0.2 | 1.3 | 4.8 | 4.6 |
| SUVR90 | −0.5 | 1.7 | 5.1 | 6.9 |
| SUVR60 | −0.5 | 1.8 | 4.9 | 6.9 |

*AD vs. Control

The PON region is not expected to differ between AD and control subjects, and thus has an effect size that varies about zero. Significant group differences in PIB retention were detected between AD and control groups for all regions except SWM and PON.

F. Discussion

In the discussion that follows, four levels of simplification will be examined: 1) shortening the scan period from 90 to 60 min; 2) substituting an arterial input function derived from a volume-of-interest defined over the carotid artery for arterial plasma-based input (CAR60/90); 3) replacing arterial input completely with an image-driven analysis method, such as the non-invasive Logan analysis (CER60/90) and SRTM90; and 4) use of a late single-scan measure of the radioactivity distribution (SUVR60/90). Within each level of simplification, performance compared to the benchmark quantitative method, ART90, was assessed by four criteria: a) fidelity of regional rank order; b) test-retest variability; c) % bias and correlation; and d) Cohen's effect size. It is acknowledged that the ART90 method is a “relative” benchmark, as there are currently no post-mortem measures of the true amyloid deposition in these subjects against which different measures of PIB retention can be independently compared.

With the exception of SRTM60 noted below, all other methods of simplification maintained regional rank order very well. Therefore, rank order will not be discussed individually in the sections below. Test-retest variability relates to the ability to detect small changes over time in amyloid deposition (in natural history studies) or amyloid clearance (in anti-amyloid therapy trials). Methodologic bias in this study was defined as the difference in outcome measures of a simplified method to the ART90 outcome measure, normalized to the ART90 value. Effect size is an indication of the ability of a method to detect small but statistically significant differences in amyloid deposition between groups.

Shortened Scan Interval:

The first level of simplification examined the possibility of acquiring the PIB PET scan for a shorter period of time, 60 min rather than 90 min. In general, analysis methods that used 90 min of emission data performed somewhat better, although methods that used 60 min of emission data yielded useful data as judged by the evaluation criteria employed. The most notable exception was the application of SRTM using 60 min of data, which resulted in spurious values, high intersubject variability, and aberrations of regional rank order. A shorter scan duration was associated with substantially higher test-retest variability in the case of ART60 and CAR60 (Table 6), although for ART60 this measure was still within the +10% margin generally considered acceptable for most PET radiotracers (Smith, G. S. et al. Synapse, 1998; 30(4): 380-92; Volkow, N. D. et al. J Nucl Med, 1991; 34: 609-13). This is of greatest concern for longitudinal studies that require reliable repeat measures of PIB retention. Truncation to 60 min did not result in a significant change in the level of methodologic bias for CAR60 or SUVR60, but CER60 showed a larger negative % bias compared to CER90 (FIG. 16A). Inter-subject variability was only substantially higher in the ART60 and CAR60 datasets in SWM, which is likely a result of the failure to reach tissue:plasma equilibrium in this brain region. In general, effect size was not adversely affected by truncation of the dataset to 60 min (Table 7).

Carotid VOI-Derived Arterial Input Function:

The next level of simplification sought to obviate arterial line placement in favor of an input function derived from a volume-of-interest defined over the carotid artery on the early frames of the reconstructed PIB image. While this method is limited in that it does not provide an estimate of the unchanged fraction of PIB in plasma on an individual basis, the use of a population average metabolite correction represented a satisfactory substitute for individual data. Of all methods examined, the 90 min carotid-based method (CAR90) provided PIB DVR estimates which most closely reflected ART90 DVR values and were the least biased relative to ART90 for both low- and high-DVR subjects (FIGS. 16A and 17A). The CAR90 results were very comparable to ART90 in terms of test-retest variability (6.9% and 7.1%, table 6) and effect size (5.1 and 5.3, respectively (table 7)). The relatively high variability may relate to difficulties in accurately drawing the small carotid ROI without variable partial volume effects. However, similar variability in the ART90 (6.9%) suggests that there are either inherent sources of variability in the arterial data or that the cerebellar methods, which show the lowest test-retest variability, blunt the true variability in some way. Despite the fact that the ART90 and CAR90 effect sizes were among the smallest of the nine methods studied in this work (mostly due to higher standard deviations of the group means), these methods yielded very robust group differences that effectively distinguished AD and control subject groups. The two methods may share inaccuracies generated by the use of arterial-based metabolite corrections, although the influence of these inaccuracies should be minimized by the use of the population average metabolite correction in the CAR90 method. In addition, both ART90 and CAR90 methods are susceptible to any artifacts induced by unusual peripheral metabolism in an individual subject (see discussion below for C-6). The close agreement between ART90 and CAR90 in terms of these performance criteria indicates that a carotid region-of-interest method with a population-average metabolite correction can provide an accurate estimation of DVR measure despite the approximately 2-fold overestimation of DV values calculated by the carotid-based methods, as indicated by the cerebellar DV values reported in Table 4 as well as those calculated for all other regions (data not shown).

Reference Tissue-Based Input Function:

A further simplification is realized when estimates of the arterial input function are obviated in favor of a completely image-driven analysis method, such as the non-invasive Logan analysis (CER60, CER90) and SRTM, which rely on the identification of a consistent tissue region devoid of radiotracer specific binding, such as the cerebellum (Logan, J. et al. J Cereb Blood Flow Metab, 1996; 16(5): 834-40; Lammertsma, A. A. et al. Neuroimage, 1996; 4(3 Pt 1): 153-8.). The CER60 and CER90 methods resulted in DVR estimates that were negatively biased with respect to ART90 DVR measures (FIG. 16), particularly in high-binding subjects. This bias appeared to be unrelated to the tissue efflux constant, k₂, because it remained whether or not k₂ was constrained to the population average k₂ value, k̅₂, in the determination of the CER90DVR (data not shown). As suggested by Logan et al., (1996), supra, the k̅₂ constraint in the non-invasive Logan analysis may be omitted without resulting in a significant aberration of the DVR measure when the ratio of the target to reference tissue radioactivity concentration (C(t)/Cᵣ(t)) remains constant for a protracted period. For PIB, this condition appears to be satisfied, as evidenced by stable tissue:cerebellar ratios after 45 min in high-DVR regions (FIG. 13). The negative bias observed in high-DVR subjects using cerebellar-input methods may be attributable to an increased influence of cerebellar pharmacokinetics, compared to plasma-based methods which use cerebellar outcome measures only to normalize regional outcome measures for the computation of DVR. This effect appears to be less important in subjects with lower levels of amyloid deposition. Previous fully-quantitative PIB studies showed that the cerebellar data were inadequately described by a 1-tissue (2 parameter) compartment model and required 2 tissue compartments. Although this fact raises concern regarding the application of SRTM for the analysis of PIB data, SRTM DVR values were slightly less biased in high-binding subjects compared to CER90, and considerably less biased relative to CER60.

The non-invasive Logan methods (CER60 and CER90) had the lowest test-retest variability of any method examined, averaging +4.4% and +4.6% across all regions, respectively. SRTM90 showed slightly higher test-retest variability (+6.2% across regions) than CER60 or CER90, though this level of variability would be considered to represent a satisfactory level of performance for a PET imaging agent. Inter-subject variability in the control group was substantially higher for SRTM90 than either CER60 or CER90, though in the AD group the methods were more comparable. This fact largely explains the larger effect sizes observed for CER60 and CER90 compared to SRTM90.

Late Single Scan Measure:

The greatest degree of simplification is realized using a method based on a late single-scan measure of the static radioactivity distribution, such as the SUV-based methods (SUVR90 and SUVR60). These assessments do not require the collection of a complete dynamic emission dataset or arterial input function data. Rather, they are based solely on regional differences in the distribution of radioactivity in the brain over some later time interval following radiotracer injection, after which specific binding of radiotracer is expected to be a major component of brain radioactivity concentrations. Because of its simplicity, the SUV measure is frequently employed in clinical studies where it can be impractical to employ quantitative analysis methods that require dynamic imaging or input function determination. To eliminate a major source of variability in the determination of SUV, the time interval for the evaluation of the SUV parameter must be chosen such that the change in the SUV value over the interval is relatively small in comparison to the SUV value itself (Beaulieu, S. et al. J Nucl Med, 2003; 44(7): 1044-50).

In the case of in vivo PET studies, the SUVR reflects the relative contributions of specific and non-specific binding to the measured signal and is therefore more comparable to the DVR value, which has been corrected for non-specific binding by normalizing regional DV estimates with the cerebellar DV value. For the PIB data, the ratio of tissue (amyloid containing) to cerebellar radioactivity was relatively constant beyond 40 min post-injection in both AD an control subjects (FIG. 13), and therefore consistent with the determination of the SUV ratios after this time. The ratio also eliminates other sources of variability such as body composition and inaccuracies in determining the injected dose (e.g., partial extravasation), which may adversely impact the calculation of SUV (Thie, J. A. J Nucl Med. 2004; 45(9): 1431-4.). Both SUVR90 and SUVR60 showed strong positive biases in PCG relative to ART90 that were similar in the low- and high-DVR subjects (FIG. 16A). Test-retest variability for the SUVR methods was among the lowest of the methods examined, second only to CER90 and CER60. Inter-subject variability was comparable between SUVR60 and SUVR90 and similar to the low variability observed in controls for the CER60 and CER90 methods. In AD subjects, the SUV methods showed a level of variability that was consistent with other methods. The SUV based methods showed the greatest dynamic range and mean difference between control and AD subjects of all methods examined, which coupled with reasonably low variability, produced the largest effect sizes of any method as well (Table 7).

Selecting a Method-of-Choice:

The selection of a method-of-choice will depend upon the nature of the particular application. All of the simplified methods examined in this study provided results that compared well to the ART90 method and overall the similarities were greater than the differences between methods. Nevertheless, each method has certain advantages and disadvantages for specific purposes.

Scan Duration:

In general, it appears that all methods that use 90 min of data consistently outperform the corresponding method using only the first 60 min. Acquisition of this data requires a full 90 min dynamic scan for CER90, CAR90, SRTM90, but all of the data necessary for the SUVR90 analysis can be obtained by having the subject in the scanner during just the 40-90 min time window. The comparable performance of the SUVR60 method using the 40-60 min window suggests that it may be possible to optimize/shorten the 40-90 min window even further without loss of performance. This may be especially important for the study of severe AD patients who may not be able to tolerate a full 90 min of emission data acquisition. In addition to the shorter scan time, other advantages of the SUVR method include simplicity of application (making it more applicable to routine clinical studies), superior PCG effect size (6.9), very good test-retest reproducibility (5.0%) and a large dynamic range (evidenced by a positive bias vs. ART90). A disadvantage shared by the SUVR, CER and SRTM methods is greater influence of any inaccuracies contributed by the cerebellar data used as reference. This would be particularly apparent if there was detectable amyloid deposition in the cerebellum.

Cross-Sectional Inter-Group Comparisons:

In the primary areas-of-interest (e.g. PCG, FRC, PAR), all methods demonstrated the ability to distinguish AD and control subjects without any overlap between groups (FIG. 4). Although the exploratory effect size calculations yielded greatest d values for the SUV-based methods (d=6.9 for PCG), even the lowest PCG effect size of 4.6 observed using the SRTM90 method is a very large effect given the definition of Cohen that any effect size>0.8 is considered “large” (Cohen, J. Statistical power for the behavioral sciences. 2nd ed. 1988, Hillsdale, N.J.: Erlbaum.). To put this in another perspective, using a parametric test of significance (2-tailed t-test), even the lowest PCG effect size of 4.6 corresponds to a highly significant difference between the AD and control group mean values with a p value of <0.0000001. Effect sizes were also calculated in MTC, an area with low amyloid deposition, and while this is not likely to be an area of focus for PIB studies in general, it may offer some indication of how high-amyloid areas, such as PCG and FRC, behave at very early stages. Effect size in the MTC was largest for ART90 (1.9), but CER90 (1.8), SUVR60 (1.8), and CAR90 (1.6) performed similarly.

Studies that Correlate Amyloid Load with Other Variables:

For some purposes, it may be important to distinguish subjects across a large spectrum of amyloid deposition and perhaps correlate amyloid deposition with other variables (e.g., neuropsychological measures, regional FDG or MRI measures, blood or CSF measures of amyloid). A biased but reliable method could provide DVR values that are restricted in dynamic range or erroneously distributed depending on the uniformity of bias. Thus statistical correlation of the PIB retention measures with other indices could be limited when the degree of bias is not uniform across the range of expected values, as is the case with the CER60 and CER90 methods. An additional difficulty that could arise in relating other variables to measures of amyloid deposition is the lack of normally distributed data, especially when all subject groups are combined. Since most measures of correlation, most notably Pearson's and Spearman's, are based on the assumption of bivariate normal distributions, correlations would only be accurate within subgroups which appear to be normally distributed. This is perhaps of paramount concern for the study of MCI subjects, which is a heterogeneous group of subjects that spans the entire range of PIB retention, but has a distinctly bimodal distribution. The study of MCI subjects is perhaps one of the more interesting and promising applications of PIB, as there are no effective non-invasive indicators of progression of amyloid pathology. In this situation, it would be advantageous to apply the simplified method with the lowest and most uniform bias (e.g., CAR90) although at the expense of higher test-retest variability.

Longitudinal Studies:

A third type of comparison study is one in which longitudinal examinations of PIB retention are made in the same subject to study the natural history of disease progression or the response to anti-amyloid therapies. In this instance, it is desirable to have the most reliable repeat measure possible in order to be sensitive to what could potentially be small changes in the degree of amyloid deposition or resorption between serial examinations. This may be an important consideration when planning a longitudinal study using PIB where one would expect the differences in PIB retention between serial examinations to be small, or a study which focused on MCI or normal aging where there would be the expectation of a lower specific binding signal. While the cerebellar methods CER90 and CER60 have shown the lowest test-retest variability, one must again consider whether or not this advantage is offset by the inherent bias in these methods. However, the low test-retest variability makes CER90 an attractive method for detecting small effects of experimental anti-amyloid therapies over time, particularly in cases with low levels of amyloid deposition that must ultimately be the principle target of these therapies.

In summary, when it is not possible or desirable to obtain arterial-based input data, several simplified methods can be valid alternatives to quantitative arterial-based analyses. The SUVR90 method may be the method of choice when simplicity of calculations and short in-scanner time are the overriding concerns. The CAR90 method may be the method of choice when comparison across a large range of amyloid deposition and minimization of cerebellum-derived artifacts is the prime concern. The CER90 method may be the method of choice for natural history studies and treatment trials, particularly in subjects with lower levels of amyloid deposition, when the detection of small interval changes is paramount. SUVR90 may perform better in treatment trials in subjects at the high end of amyloid deposition. In practice, the data necessary for all of these analyses will be available after a 90-minute dynamic PIB scan, and so the decision regarding method of choice does not necessarily need to be made beforehand.

Other embodiments of the invention will be apparent to those skilled in the art from consideration of the specification and practice of the invention disclosed herein. It is intended that the specification be considered as exemplary only, with the true scope and spirit of the invention being indicated by the following claims.

As used herein and in the following claims, singular articles such as “a”, “an”, and “one” are intended to refer to singular or plural.

What is claimed is:

1. A method of identifying a patient as prodromal to a disease associated with amyloid deposition comprising:
(A) administering to the patient, who is presenting with signs of clinical dementia or clinical signs of a mild cognitive impairment, a compound of the following formula: 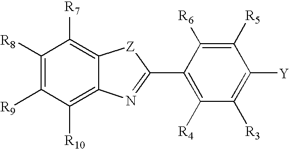
wherein
(i) Z is S, NR′, O or C(R′)₂, such that when Z is C(R′)₂, the tautomeric form of the heterocyclic ring may form an indole: 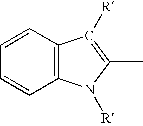
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R¹ is H or a lower alkyl group),
(iv) R² is selected from the group consisting of H, a lower alkyl group, (CH₂)ₙOR′ (wherein n=1, 2 or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R′ is H or a lower alkyl group),
(v) each R³-R¹⁰ independently is selected from the group consisting of H, F, Cl, Br, I, a lower alkyl group, (CH₂)ₙOR′ (wherein n=1, 2 or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, with the phenyl substituents chosen from any of the non-phenyl substituents defined for R¹-R¹⁰ and wherein R′ is H or a lower alkyl group), a tri-alkyl tin and a chelating group (with or without a chelated metal group) of the form W-L or V—W-L, wherein V is selected from the group consisting of —COO—, —CO—, —CH₂O— and —CH₂NH—; W is —(CH₂)ₙ (where n=0, 1, 2, 3, 4 or 5) and L is: 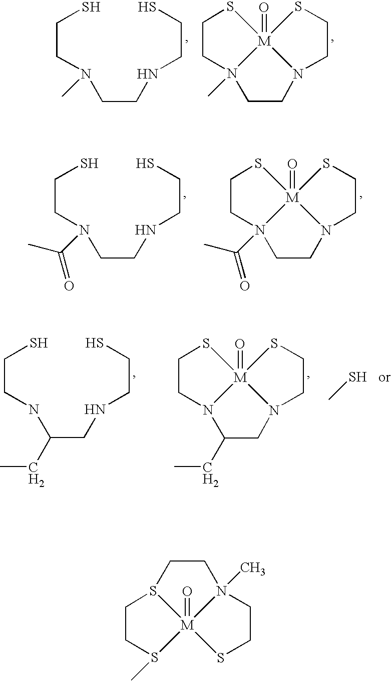
wherein M is selected from the group consisting of Tc and Re; and radiolabelled derivatives and pharmaceutically acceptable salts thereof, where at least one of the substituent moieties comprises a detectable label;
then
(B) imaging said patient to obtain data; and
(C) analyzing said data to ascertain amyloid levels in said patient with reference to a normative level, thereby identifying said patient as prodromal to a disease associated with amyloid deposition.

2. The method of claim 1, wherein the patient is diagnosed with mild cognitive impairment.

3. The method of claim 1, wherein the amyloid disease is Alzheimer's disease.

4. The method of claim 1, wherein the imaging is selected from the group consisting of gamma imaging, magnetic resonance imaging and magnetic resonance spectroscopy.

5. The method of claim 4, wherein the imaging is done by gamma imaging, and the gamma imaging is PET or SPECT.

6. The method of claim 1, wherein the compound of Formula (I) is:

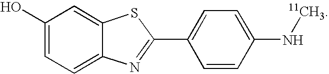

7. The method of claim 1, wherein the compound of Formula (I) contains a C-11 label.

8. The method of claim 1, where said data define a dementing disorder of questionable etiology as being caused by an amyloid deposition disease.

9. The method of claim 8, comprising distinguishing Alzheimer's disease from frontotemporal dementia.

10. The method of claim 2, further comprising monitoring said patient to determine onset of Alzheimer's disease.

11. The method of claim 1, which comprises diagnosing Alzheimer's disease in a patient clinically diagnosed with mild cognitive impairment.

12. The method of claim 1, wherein the disease associated with amyloid deposition is Alzheimer's disease.

13. The method of claim 1, wherein the patient is presenting with a dementing disorder of questionable etiology.

14. The method of claim 13, wherein the patient has undiagnosed AD.

15. The method of claim 1, wherein the patient has undiagnosed AD.

16. A compound of the following formula: 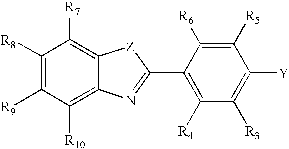
wherein
(i) Z is S, NR′, O or C(R′)₂, such that when Z is C(R′)₂, the tautomeric form of the heterocyclic ring may form an indole: 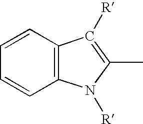
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R′ is H or a lower alkyl group),
(iv) R² is selected from the group consisting of H, a lower alkyl group, (CH₂)ₙOR′ (wherein n=1, 2 or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3 or 4 and Rₚₕ represents an unsubstituted or substituted phenyl group, with the phenyl substituents chosen from any of the non-phenyl substituents defined below for R³-R¹⁰ and R′ is H or a lower alkyl group),
(v) each R³-R¹⁰ independently is selected from the group consisting of H, F, Cl, Br, I, a lower alkyl group, (CH₂)ₙOR′ (wherein n=1, 2 or 3), CF₃, CH₂—CH₂X, O—CH₂—CH₂X, CH₂—CH₂—CH₂X, Q-CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group, with the phenyl substituents chosen from any of the non-phenyl substituents defined for R¹-R¹⁰ and wherein R′ is H or a lower alkyl group), a tri-alkyl tin and a chelating group (with or without a chelated metal group) of the form W-L or V—W-L, wherein V is selected from the group consisting of —COO—, —CO—, —CH₂O— and —CH₂NH—; W is —(CH₂)ₙ (where n=0, 1, 2, 3, 4 or 5) and L is: 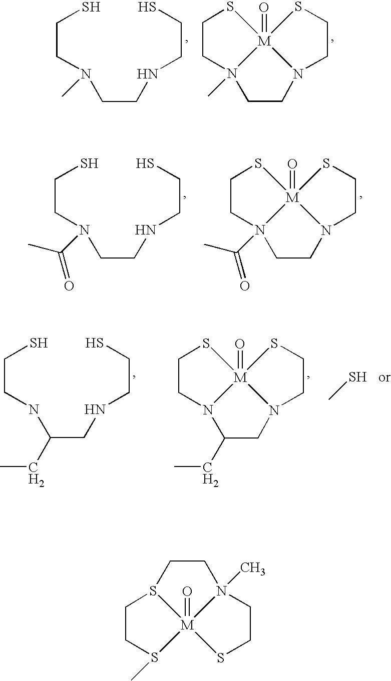
wherein M is selected from the group consisting of Tc and Re; and radiolabelled derivatives and pharmaceutically acceptable salts thereof, where at least one of the substituent moieties comprises a detectable label; for the manufacture of a medicament of a diagnostic agent for use in diagnosing a patient as prodromal to a disease associated with amyloid deposition.

17. A method of diagnosing a patient as prodromal to a disease associated with amyloid deposition comprising:
(A) imaging the patient, wherein the patient is presenting with signs of clinical dementia or clinical signs of a mild cognitive impairment, who has been administered a compound of claim 16, to obtain data; and
(B) analyzing said data to ascertain amyloid levels in said patient with reference to a normative level, thereby identifying said patient as prodromal to a disease associated with amyloid deposition.

18. The method of claim 1, wherein the detectable label is a radiolabel.

19. A method of identifying a patient as prodromal to a disease associated with amyloid deposition comprising:
(A) administering to the patient, who is presenting with clinical signs of dementia or clinical signs of a mild cognitive impairment, an amyloid imaging agent of formula (II) 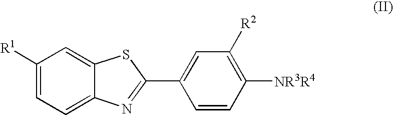
or a radiolabeled derivative, pharmaceutically acceptable salt, hydrate, solvate or prodrug of the compound, wherein:
R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;
R is C₁-C₆ alkyl;
R² is hydrogen or halo;
R³ is hydrogen, C₁-C₆ alkyl, C₂-C₆ alkenyl or C₂-C₆ alkynyl; and
R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;
provided that when R¹ is hydrogen or —OH, R² is hydrogen and R⁴ is −1 CH₃, then R³ is C₂-C₆ alkyl, C₂-C₆ alkenyl or C₂-C₆ alkynyl; and
further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label;
then
(B) imaging said patient to obtain data; and
(C) analyzing said data to ascertain amyloid levels in said patient with reference to a normative level, thereby identifying said patient as prodromal to a disease associated with amyloid deposition.

20. The method of claim 19, wherein the detectable label is a radiolabel.

21. The method of claim 1, where the amyloid imaging agent of formula (I) is selected from the group consisting of structures 1-45 or a radiolabeled derivative thereof, wherein the compound comprises at least one detectable label:

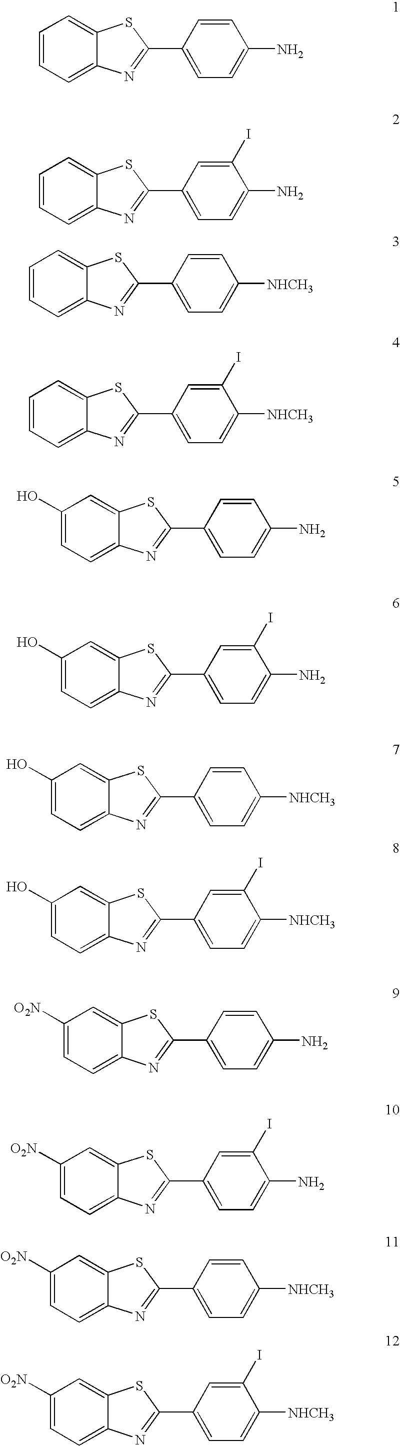

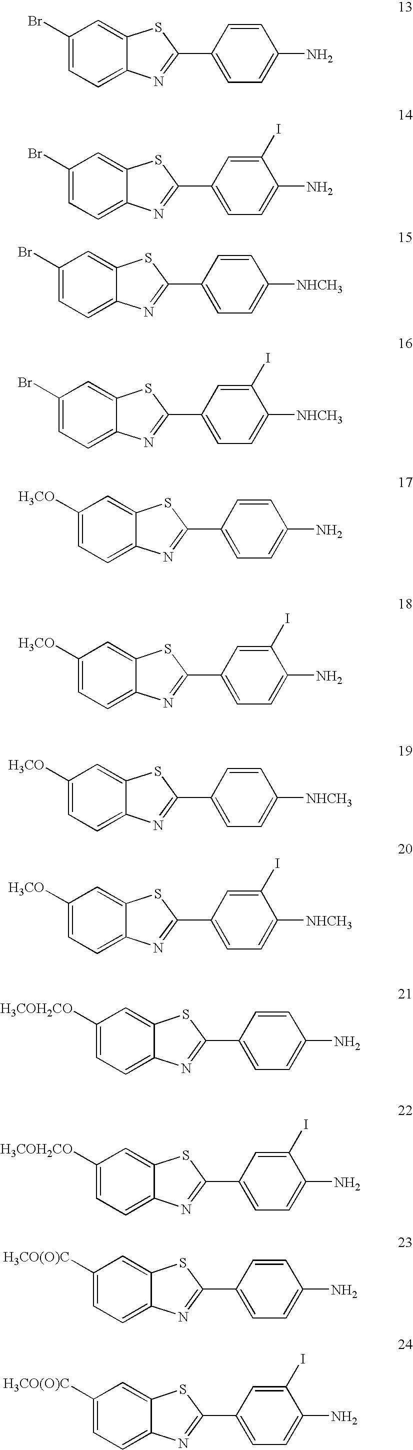

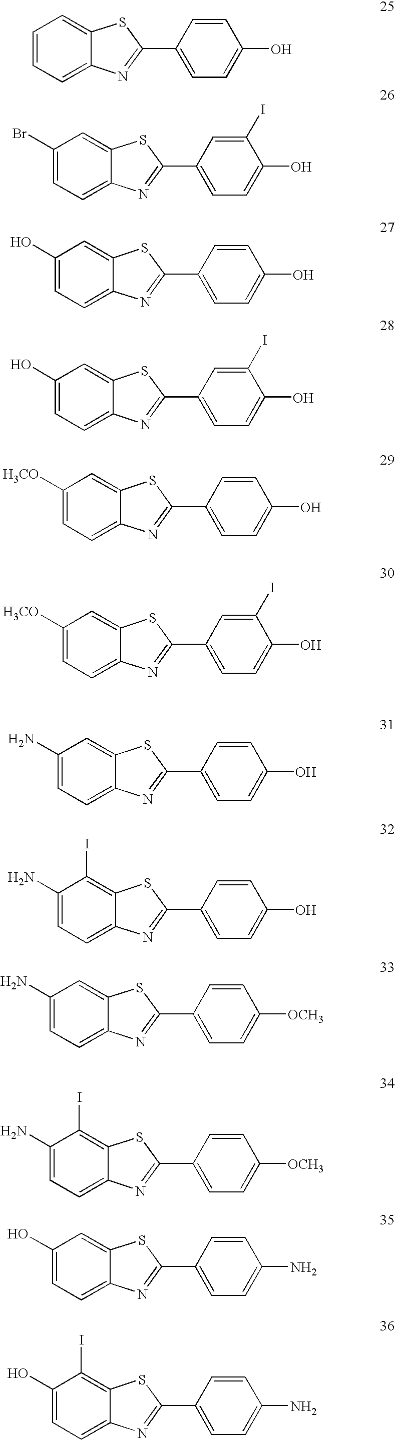

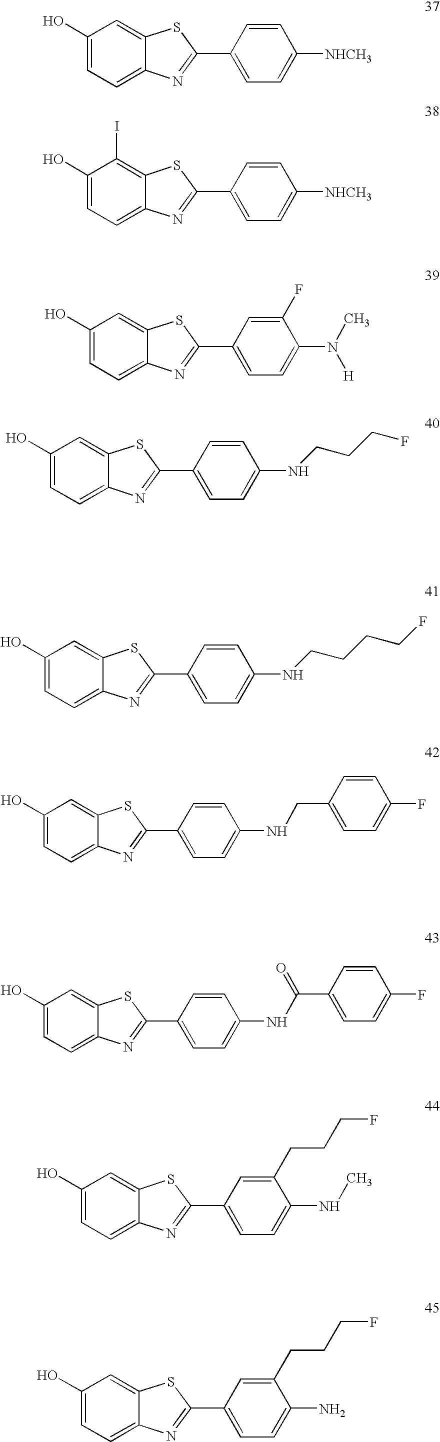

22. A compound of Formula (II): 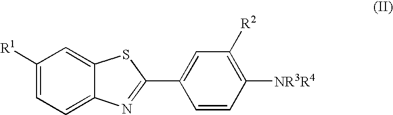
or a radiolabeled derivative, pharmaceutically acceptable salt, hydrate, solvate or prodrug of the compound, wherein:
R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;
R is C₁-C₆ alkyl;
R² is hydrogen or halo;
R³ is hydrogen, C₁-C₆ alkyl, C₂-C₆ alkenyl or C₂-C₆ alkynyl; and
R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;
provided that when R¹ is hydrogen or —OH, R² is hydrogen and R⁴ is —¹¹CH₃, then R³ is C₂-C₆ alkyl, C₂-C₆ alkenyl or C₂-C₆ alkynyl; and
further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label, for the manufacture of a medicament of a diagnostic agent for use in diagnosing a patient as prodromal to a disease associated with amyloid deposition.

23. The compound of claim 16, wherein the compound is one of structures 1-45: 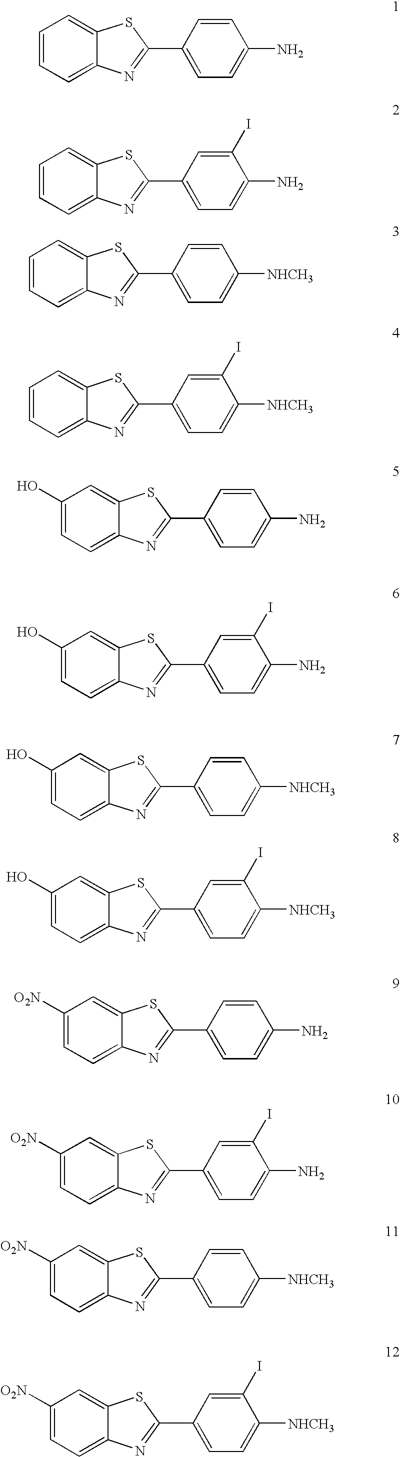 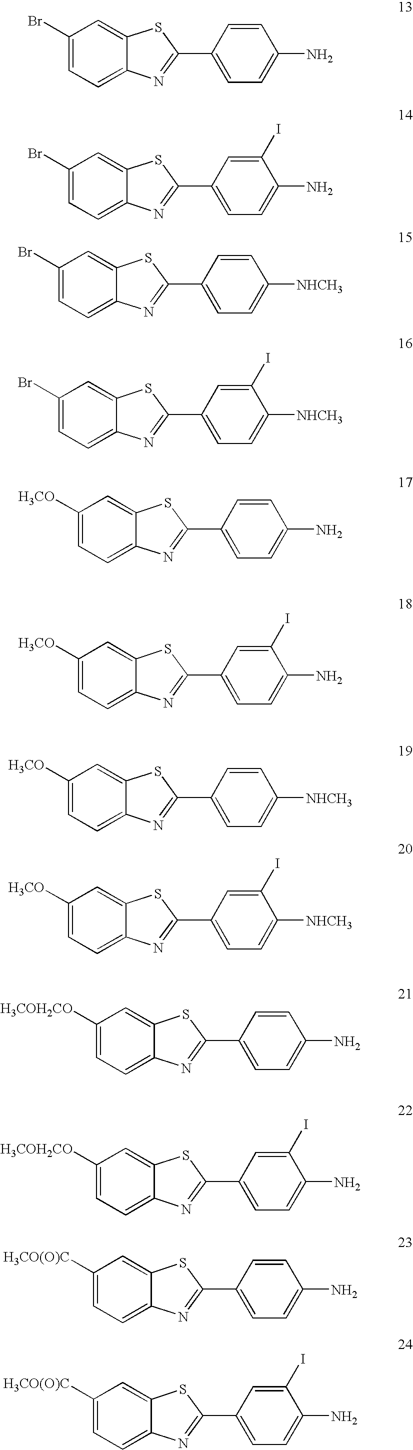 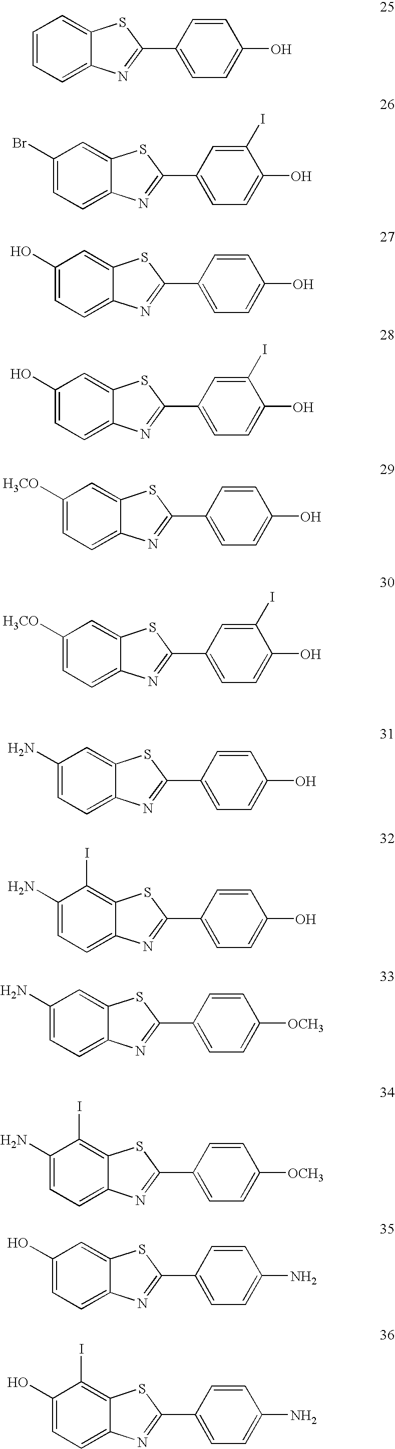 [chemical structure]
